(19) United States
(12) Patent Application Publication
(10) Pub. No.: US 2016/0108431 A1
(43) Pub. Date: Apr. 21, 2016

(54) PREVENTION AND TREATMENT OF ATRIAL FIBRILLATION

(71) Applicant: ISIS INNOVATION LIMITED, Summertown, Oxford Oxfordshire (GB)

(72) Inventors: Barbara Casadei, Oxford (GB); Svetlana Reilly, Oxford (GB)

(73) Assignee: ISIS Innovation Limited, Summertown, Oxford, Oxfordshire (GB)

(21) Appl. No.: 14/895,468

(22) PCT Filed: Jun. 3, 2014
(86) PCT No.: PCT/GB2014/051695

§ 371 Date: Dec. 2, 2015

(30) Foreign Application Priority Data
Jun. 3, 2013 (GB) 1309882.7

(51) Int. Cl. C12N 15/86

(52) U.S. Cl. CPC C12N 15/86; C12N 2710/24043; C12N 2710/10043; C12N 2750/14143

(57) ABSTRACT

There is provided a nucleic acid that inhibits miR-31 in an atrial myocyte, for use in the prevention or treatment of atrial fibrillation in a subject. Also provided is a method for diagnosing or predicting the risk of atrial fibrillation in a subject, said method comprising: (i) determining the amount of miR-31 in a sample from the subject; (ii) comparing the amount of miR-31 in the sample with a reference standard; and (iii) identifying a difference in the amount of miR-31 in the sample relative to the reference standard; wherein an increase in the amount of miR-31 in the sample compared to the reference standard correlates with the presence of or an increased risk of atrial fibrillation in the subject; and wherein a decrease, or no difference in the amount of miR-31 in the sample compared to the reference standard correlates with the absence of an increased risk of atrial fibrillation in the subject.

This patent application claims priority to GB 1309882.7 filed on 3 Jun. 2013, which is hereby incorporated by reference in its entirety.

The work leading to this invention has received funding from the European Community's Seventh Framework Programme (FP7/2007-2013) under grant agreement No. 261057.

The present invention relates to therapeutics and corresponding therapies for use in the prevention and treatment of atrial fibrillation. The present invention also relates to methods for diagnosing and predicting atrial fibrillation.

Atrial fibrillation (AF) is a heart rhythm disturbance, characterised by rapid, irregular electrical and mechanical activation of the atria, which causes uncoordinated contraction and favours the formation of atrial thrombi.

AF is the most common sustained cardiac arrhythmia and an increasing health-care burden because of the ageing population and improved survival from acute cardiovascular events such as myocardial infarction. The lifetime risk for development of AF in all men and women older than 40 is estimated at about 25%, whereas for those without previous or concurrent cardiovascular events the lifetime risk is still about 16%. The presence of AF independently increases the risk of mortality and morbidity, mostly due to stroke and heart failure, resulting in a high health-care cost.

In the United Kingdom, findings from the Screening for AF in the Elderly (SAFE) study showed a baseline prevalence of AF of 7.2% in patients aged 65 years and older, with a slightly increased prevalence in men (7.8%) and in those aged 75 years and older (10.3%), and a yearly incidence of about 1.6%. A community survey reported a rise in the incidence of AF of 12.6% during the past two decades, and projected that 15.9 million people in the USA will have the disorder by 2050.

To date, the mechanisms responsible for the new onset of AF are only partially understood, and even less is known of the processes that underlie the progression from paroxysmal to persistent AF and influence the response to treatment. In the absence of therapeutic approaches targeting the signalling pathways involved in the substrate that supports AF, current management is mainly focussed on relieving symptoms and preventing embolic stroke. Restoration of sinus rhythm by using conventional anti-arrhythmic drugs is seldom successful and can lead to life-threatening pro-arrhythmic effects in the ventricular myocardium. Overall, this and other approaches (e.g. radio-frequency ablation) have not decreased morbidity or mortality in patients with AF.

There is therefore a need for new therapeutics and associated therapies and methods that can be used to treat AF or to prevent the occurrence or development of AF in an at-risk subject. In particular, there is a need for new therapeutic interventions against AF that target the underlying molecular mechanisms rather than merely treating symptoms.

The present invention solves one or more of the above problems by providing new therapeutics for use in preventing or treating AF in a subject (such as a human subject), together with corresponding methods for preventing or treating AF. The present invention also provides methods of diagnosing or predicting AF.

In one aspect, the invention provides a nucleic acid that inhibits miR-31 in an atrial myocyte, for use in the prevention or treatment of AF in a subject.

In a related aspect, the invention provides a method of preventing or treating AF in a subject, comprising administering to the subject an effective amount of a nucleic acid that inhibits miR-31 in an atrial myocyte. As used herein, an “effective amount” is a dosage or amount that is sufficient to achieve the desired biological outcome of the prevention or treatment of AF.

The present inventors have found that inhibition of miR-31 using a nucleic acid miR-31 inhibitor can prevent or treat AF. Without wishing to be bound by any particular theory, the inventors believe that an atrial-specific upregulation of miR-31 in AF is responsible for a reduction in dystrophin and syntrophin content in the fibrillating atrial myocardium, with an associated decrease in nNOS (neuronal nitric oxide synthase) protein levels and a concomitant depression of atrial nNOS function. This in turn decreases atrial myocyte nNOS signalling and creates an atrial electrical substrate that increases both the inducibility and maintenance of AF.

Inhibition of miR-31 by administration of a nucleic acid miR-31 inhibitor can therefore block the above-described miR-31-dependent processes and so be used to prevent AF from occurring in an otherwise susceptible (i.e. at-risk) subject, or to treat AF in a subject by reducing the frequency, duration and/or magnitude of AF episodes. In the present context, administration means that the nucleic acid miR-31 inhibitor is applied to the subject such that it reaches the atrial myocytes.

MiR-31 is a microRNA. MicroRNAs are endogenous, small (about 19-25 nucleotides [nt]), non-protein coding RNAs that bind to complementary sequences in mRNA transcripts, inducing gene silencing via mRNA degradation or translational repression. For example, complete complementarity of the mature microRNA sequence to the target mRNA can lead to target cleavage and inhibition of protein synthesis, while incomplete pairing can lead to translational repression either through mRNA destabilisation or through removal of the 5′ cap or 3′ poly-A termination signal. MicroRNAs are known to regulate a wide variety of biological processes, including metabolism, cell proliferation and differentiation, and apoptosis. It has also been theorised that microRNAs play a role in the development of certain diseases, such as cancer.

The microRNA miR-31 is 21 nt in length and has the sequence AGGCAAGAUGCUGGCAUAGCU (SEQ ID NO: 2); this represents the sequence of the mature miR-31. MiR-31 has Gene ID: 407035, and miRBase accession number MIMAT0000089.

A mature microRNA sequence is produced by processing of initial primary microRNA (pri-miRNA) sequences expressed from the genome. The sequence of miR-31 is produced from an earlier precursor sequence (encompassing the final miR-31 sequence):

GGAGAGGAGGCAAGAUGCUGGCAUAGCUGUUGAACUGGGAACCUGCUA UGCCAACAUAUUGCCAUCUUUCC (SEQ ID NO: 3).

Prior to the present invention, no role for miR-31 in atrial fibrillation has been known. The present inventors have discovered that inhibition of miR-31 in atrial myocytes restores both dystrophin and nNOS content, leading to changes in atria electrical properties that reduce new onset and maintenance of AF, and lead to a reduction in the incidence and symptoms of AF.

In the present context, inhibition of miR-31 means that the biological activity of miR-31 is reduced (i.e. inhibited). Thus, inhibition of miR-31 reduces or abolishes the biological effects of miR-31 activity. The present inventors have discovered that miR-31 inhibition can be effected in the human atrial myocardium using a nucleic acid miR-31 inhibitor.

As described herein, a nucleic acid miR-31 inhibitor (i.e. a nucleic acid that inhibits miR-31) is any nucleic acid, for example an oligonucleotide, that reduces (i.e. inhibits) the biological activity of miR-31. In preferred embodiments, nucleic acid miR-31 inhibitors comprise a nucleic acid sequence that is complementary with at least a portion of the nucleic acid sequence of either miR-31 itself or the target mRNA sequences of miR-31; inhibition of miR-31 therefore occurs by binding of the inhibitor to miR-31 or to its target mRNA. In both cases, miR-31 is prevented from recognising and binding its target sequence and thus cannot induce gene silencing. The nucleic acid miR-31 inhibitor of the invention may comprise a structure that is single stranded, double stranded, partially double-stranded, or hairpin in nature.

In one embodiment, the nucleic acid miR-31 inhibitor (as described above), binds to miR-31. Thus, in one embodiment, the nucleic acid miR-31 inhibitor binds to and sequesters miR-31 in atrial myocytes, so preventing it from binding to its target mRNA sequences. Binding occurs via complementary base pairing between at least one nucleotide present in the nucleic acid miR-31 inhibitor and a corresponding nucleotide present in miR-31, such that at least a portion of the nucleic acid miR-31 inhibitor and miR-31 together define a base-paired nucleic acid duplex. Said complementary base pairing (and thus duplex formation) can occur over a region of two or more contiguous nucleotides of miR-31 (e.g. 3, 4, 5, 6, 7, 8, 9, 10, 11, 12, 13, 14, 15, 16, 17, 18, 19, 20 or 21 contiguous nucleotides). A base-paired nucleic acid duplex formed when the nucleic acid miR-31 inhibitor binds to miR-31 (as described above) may comprise one or more mismatch pairings. In certain embodiments, two or more regions of complementary base-paired nucleic acid duplex (e.g. 3, 4, 5 or 6) are formed, wherein each region is separated from the next by one or more mismatch pairings.

As used herein, the term “complementary” refers to a nucleic acid molecule that forms hydrogen bonds with another nucleic acid molecule with Watson-Crick base pairing. Watson-Crick base pairing refers to the following hydrogen bonded nucleotide pairings: A:T and C:G (for DNA); and A:U and C:G (for RNA). For example, two or more complementary nucleic acid molecule strands can have the same number of nucleotides (i.e. have the same length and form one double-stranded region, with or without an overhang) or have a different number of nucleotides (e.g. one strand may be shorter than but fully contained within another strand or one strand may overhang the other strand).

Mismatch pairings are formed between any two nucleotide bases that together do not form one of the hydrogen-bonded standard Watson-Crick base pairs of A:U (in RNA), A:T (in DNA) and C:G (in both RNA and DNA).

In one embodiment, the nucleic acid miR-31 inhibitor comprises (or consists of) a nucleic acid sequence complementary to at least a portion of the miR-31 sequence. The nucleic acid miR-31 inhibitor may comprise a nucleic acid sequence complementary to 2, 3, 4, 5, 6, 7, 8, 9, 10, 11, 12, 13, 14, 15, 16, 17, 18, 19, 20 or 21 contiguous nucleotides of miR-31. In one embodiment, the nucleic acid miR-31 inhibitor comprises (or consists of) a nucleic acid sequence complementary to the sequence AGGCAAGAUGCUGGCAUAGCU (SEQ ID NO: 2).

In one embodiment, the nucleic acid miR-31 inhibitor comprises (or consists of) a nucleic acid sequence that binds (such as by complementary binding) to at least a portion of the miR-31 seed region, wherein said seed region comprises the nucleotides at positions 2-7 of miR-31. Research has shown that the “seed” region of a microRNA, which comprises the nucleotides at positions 2-7 (also referred to as nucleotides 2-7, said positions counted from the 5′ end of the microRNA nucleic acid, wherein the first nucleotide at the 5′ end is position 1), plays an important role in the binding of a microRNA to its mRNA target. In one embodiment, the seed region comprises the nucleotides at positions 2-8 of miR-31. Thus, the binding of a nucleic acid miR-31 inhibitor to at least a portion of said seed region prevents the seed region from being able to recognise and bind its target mRNA.

In one embodiment, said portion of the miR-31 seed region comprises at least five of the nucleotides at positions 2-7 of miR-31.

In one embodiment, the nucleic acid miR-31 inhibitor comprises (or consists of) a nucleic acid sequence that binds (such as by complementary binding) to the nucleotides at positions 2-7 of miR-31 with no more than 2 (e.g. no more than 2, or no more than 1) mismatches.

In one embodiment, the nucleic acid miR-31 inhibitor comprises (or consists of) a nucleic acid sequence that binds (such as by complementary binding) to the six nucleotides at positions 2-7 of miR-31. In one embodiment, the nucleic acid sequence also binds to the nucleotide at position 8 of miR-31.

In one embodiment, the nucleic acid miR-31 inhibitor comprises the nucleic acid sequence CUUGCC (SEQ ID NO: 4). Said sequence represents the complementary sequence (written in the direction 5′-3′) to nucleotides 2-7 of miR-31. Thus, a nucleic acid miR-31 inhibitor comprising said sequence will bind by complementary binding to the seed region of miR-31.

In one embodiment, the nucleic acid miR-31 inhibitor comprises a nucleic acid sequence having at least 70% (such as at least 70, 75, 80, 85, 90, 95 or 100%) sequence identity to SEQ ID NO: 1. The nucleic acid sequence of SEQ ID NO: 1 represents the complementary (i.e. antisense) sequence of miR-31. Thus, the nucleic acid sequence of SEQ ID NO: 1 will (under ideal conditions) bind with 100% complementarity to miR-31. Thus, in one embodiment, the nucleic acid miR-31 inhibitor comprises a sequence complementary to miR-31.

In one embodiment, wherein the nucleic acid miR-31 inhibitor comprises a nucleic acid sequence that binds (such as by complementary binding) to at least a portion of the miR-31 seed region (as described above), the nucleic acid miR-31 inhibitor comprises a nucleic acid sequence having at least 70% (such as at least 70, 75, 80, 85, 90, 95 or 100%) sequence identity to SEQ ID NO: 1.

In one embodiment, wherein the nucleic acid miR-31 inhibitor comprises a nucleic acid sequence having at least 70% (such as at least 70, 75, 80, 85, 90, 95 or 100%) sequence identity to SEQ ID NO: 1, said nucleic acid sequence further comprises the sequence CUUGCC which binds (such as by complementary binding) to the six nucleotides at positions 2-7 of miR-31.

In one embodiment, the nucleic acid miR-31 inhibitor comprises (or consists of) a nucleic acid sequence that differs from the sequence of SEQ ID NO: 1 at a maximum of 10 (e.g. a maximum of 10, 9, 8, 7, 6, 5, 4, 3, 2, or 1) nucleotide positions. In one embodiment, the nucleic acid miR-31 inhibitor comprises (or consists of) a nucleic acid sequence that differs from the sequence of SEQ ID NO: 1 at a maximum of 5 (e.g. a maximum of 4, 3, 2, or 1) nucleotide positions. Thus, said nucleic acid sequence is identical to the sequence of SEQ ID NO: 1 except at a limited number of nucleotide positions. Preferably, the nucleotides that bind to nucleotides 2-7 of miR-31 are unchanged from those of SEQ ID NO: 1.

In one embodiment, wherein the nucleic acid miR-31 inhibitor comprises a nucleic acid sequence that binds (such as by complementary binding) to at least a portion of the miR-31 seed region (as described above), the nucleic acid miR-31 inhibitor comprises (or consists of) a nucleic acid sequence that differs from the sequence of SEQ ID NO: 1 at a maximum of 10 nucleotide positions, or a maximum of 5 nucleotide positions, as described above.

In one embodiment, wherein the nucleic acid miR-31 inhibitor comprises (or consists of) a nucleic acid sequence that differs from the sequence of SEQ ID NO: 1 at a maximum of 10 (e.g. a maximum of 10, 9, 8, 7, 6, 5, 4, 3, 2, or 1) nucleotide positions, or wherein the nucleic acid miR-31 inhibitor comprises (or consists of) a nucleic acid sequence that differs from the sequence of SEQ ID NO: 1 at a maximum of 5 (e.g. a maximum of 4, 3, 2, or 1) nucleotide positions, said nucleic acid sequence further comprises the sequence CUUGCC which binds by complementary binding to the six nucleotides at positions 2-7 of miR-31.

In one embodiment, the nucleic acid miR-31 inhibitor, as described above, has a maximum length of 60 (for example 55, 50, 45, 40, 35, 30, or 25) nucleotides.

In one embodiment, the nucleic acid miR-31 inhibitor comprises two or more (e.g. 3, 4, 5 or 6) nucleic acid sequences, wherein each of said two or more nucleic acid sequences has at least 70% (such as at least 70, 75, 80, 85, 90, 95 or 100%) sequence identity to SEQ ID NO: 1. Optionally, each of said two or more nucleic acid sequences may further comprise the sequence CUUGCC which binds (such as by complementary binding) to the six nucleotides at positions 2-7 of miR-31.

In another embodiment, the nucleic acid miR-31 inhibitor comprises two or more (e.g. 3, 4, 5 or 6) nucleic acid sequences, wherein each of said two or more nucleic acid sequences differs from the sequence of SEQ ID NO: 1 at a maximum of 10 (e.g. a maximum of 10, 9, 8, 7, 6, 5, 4, 3, 2, or 1) nucleotide positions, or at a maximum of 5 (e.g. a maximum of 4, 3, 2, or 1) nucleotide positions. Optionally, each of said two or more nucleic acid sequences may further comprise the sequence CUUGCC which binds (such as by complementary binding) to the six nucleotides at positions 2-7 of miR-31.

In one embodiment, the nucleic acid miR-31 inhibitor is a miR-31 antagomir. An antagomir is a nucleic acid oligomer that is designed to bind to a specific target microRNA via complementary base pairing (for example, as described above). An antagomir may have a sequence that is wholly or partially complementary to the target microRNA sequence. Antagomirs may have a single stranded, double stranded, partially double-stranded, or hairpin structure. Antagomirs may further comprise chemically modified nucleotides (e.g. as described below).

Methods for designing and creating antagomirs are known in the art. In one embodiment, the nucleic acid miR-31 inhibitor comprising a nucleic acid sequence having at least 70% sequence identity to SEQ ID NO: 1 (as described above) is a miR-31 antagomir.

In one embodiment, the nucleic acid miR-31 inhibitor is a miR-31 microRNA-sponge. A microRNA-sponge is a nucleic acid that comprises multiple (e.g. at least 2, 3, 4, 5 or 6) binding sites for a specific target microRNA. Thus, a microRNA-sponge is able to bind and sequester multiple target microRNA molecules. A microRNA sponge may comprise an mRNA expressed from a vector (e.g. a viral vector or plasmid vector). The presence in a microRNA-sponge of multiple binding sites for the target microRNA enables microRNAs to be adsorbed in a manner analogous to a sponge soaking up water.

A microRNA-sponge may bind target microRNAs via complementary base pairing (for example, as described above). Thus, a microRNA-sponge may comprise multiple (e.g. at least 2, 3, 4, 5 or 6) nucleic acid sequences, each sequence being complementary to at least a portion of the target microRNA sequence. A microRNA-sponge may comprise multiple (e.g. at least 2, 3, 4, 5 or 6) nucleic acid sequences, wherein each sequence is complementary to the target microRNA sequence. Methods for designing and creating microRNA-sponges are known in the art. In one embodiment, the nucleic acid miR-31 inhibitor comprising two or more nucleic acid sequences, wherein each of said two or more nucleic acid sequences has at least 70% sequence identity to SEQ ID NO: 1 (as described above) is a miR-31 microRNA sponge.

In one embodiment, the nucleic acid miR-31 inhibitor competes with miR-31 for binding to a miR-31 mRNA target site in an atrial myocyte. As discussed above, microRNAs bind to complementary sequences in specific target mRNAs. Thus, a miR-31 mRNA target site represents one such complementary sequence in a target mRNA.

Thus, in one embodiment, the nucleic acid miR-31 inhibitor does not bind directly to miR-31 but instead binds to a miR-31 mRNA target site. This has the effect of blocking said target site (for example, by steric interference), preventing its recognition and binding by miR-31, and thus inhibiting miR-31 and its actions. In contrast to the binding of miR-31 to an mRNA target site, the binding of the nucleic acid miR-31 inhibitor of the invention to a miR-31 mRNA target site does not induce gene silencing (e.g. by mRNA degradation or translational repression) of said target mRNA.

In one embodiment, the miR-31 mRNA target site is located on a dystrophin 3′ UTR. In one embodiment, the miR-31 mRNA target site is located on a nNOS 3′ UTR. In one embodiment, the miR-31 inhibitor is a Target Site Blocker (TSB).

Binding of the nucleic acid miR-31 inhibitor to a miR-31 mRNA target site may occur via complementary base pairing, as described above. Thus, in one embodiment, binding between the nucleic acid miR-31 inhibitor and the miR-31 mRNA target site occurs via complementary base pairing between at least one nucleotide present in the nucleic acid miR-31 inhibitor and a corresponding nucleotide present in the miR-31 mRNA target site, such that at least a portion of the nucleic acid miR-31 inhibitor and the miR-31 mRNA target site together define a base-paired nucleic acid duplex. Said complementary base pairing (and thus duplex formation) can occur over a region of two or more contiguous nucleotides of the miR-31 mRNA target site (e.g. 3, 4, 5, 6, 7, 8, 9, 10, 11, 12, 13, 14, 15, 16, 17, 18, 19, 20 or 21 contiguous nucleotides). A base-paired nucleic acid duplex formed when the nucleic acid miR-31 inhibitor binds to the miR-31 mRNA target (as described above) may comprise one or more mismatch pairings. In certain embodiments, two or more regions of complementary base-paired nucleic acid duplex (e.g. 3, 4, 5 or 6) are formed, wherein each region is separated from the next by one or more mismatch pairings.

In one embodiment, the mRNA target site is located on the 3′ UTR (untranslated region) of the target mRNA. In one embodiment, the mRNA target site is located on the dystrophin mRNA (e.g. on the 3′ UTR). In another embodiment, the mRNA target site is located on the nNOS mRNA (e.g. on the 3′ UTR).

In one embodiment, wherein the nucleic acid miR-31 inhibitor competes with miR-31 for binding to a miR-31 mRNA target site in an atrial myocyte (as described above), the nucleic acid comprises the nucleic acid sequence GGCAAG (SEQ ID NO: 5). Thus, said nucleic acid sequence will bind to the miR-31 mRNA target site via complementary binding at the location targeted by the seed region of miR-31, thus preventing miR-31 from binding.

In one embodiment, the nucleic acid miR-31 inhibitor is a small interfering RNA (siRNA) that is targeted against miR-31.

In one embodiment, the nucleic acid miR-31 inhibitor comprises (or consists of) a nucleic acid sequence selected from:

(SEQ ID NO: 6)
AGGCAAGATGCTGGCATAGCT,
(SEQ ID NO: 7)
TCCGTTCTACGACCGTATCGA,
(SEQ ID NO: 8)
TGGCAAGAATGAAAAGGC,
and
(SEQ ID NO: 9)
GGCAAGTTATTTAGCTAT.

In one embodiment, the nucleic acid miR-31 inhibitor comprises (or consists of) a nucleic acid sequence having at least 70% (such as at least 70, 75, 80, 85, 90, 95 or 100%) sequence identity to a nucleic acid sequence selected from SEQ ID NOs 6, 7, 8 and 9.

The nucleic acid miR-31 inhibitor of the invention may be a ribonucleic acid (RNA) or a deoxyribonucleic acid (DNA), or may comprise both RNA and DNA. Thus, in one embodiment, a nucleic acid miR-31 inhibitor (as described above) comprises (or consists of) RNA. In one embodiment, a nucleic acid miR-31 inhibitor (as described above) comprises (or consists of) DNA.

Unless specifically indicated otherwise, nucleic acid sequences in this document are written in the direction 5′-3′. As would be understood by a person skilled in the art, nucleic acid sequences written as RNA and containing the nucleotide U may equally be written as DNA by substituting T for U. Reference to nucleic acid(s) and/or nucleotide(s) embraces modified nucleic acid(s) and modified nucleotide(s). For example, a nucleic acid or nucleotide may be modified to increase the stability of said nucleic acid or nucleotide, for example by improving resistance to nuclease degradation. In one embodiment, a modified nucleic acid comprises a locked nucleic acid (LNA) nucleotide. In more detail, the ribose moiety of an LNA nucleotide is modified with an extra bridge connecting the 2′ oxygen and 4′ carbon. The bridge “locks” the ribose in the 3′-endo (North) conformation, which is often found in A-form nucleic acid duplexes. LNA nucleotides can be mixed with DNA or RNA residues in an oligonucleotide whenever desired. The locked ribose conformation enhances base stacking and backbone pre-organization. This significantly increases the hybridization properties (melting temperature) of oligonucleotides. Other examples of modified nucleotides include 2′-methoxyethoxy (MOE) nucleotides; 2′-methyl-thio-ethyl, 2′-deoxy-2′-fluoro nucleotides. 2′-deoxy-2′-chloro nucleotides, 2′-azido nucleotides, and 2′-O-methyl nucleotides. A nucleic acid molecule of the invention may also be conjugated to one or more cholesterol moieties. Thus, in one embodiment, the nucleic acid miR-31 inhibitor is conjugated to at least one (for example, 1, 2, 3, or 4) cholesterol moiety.

Thus, in one embodiment, the nucleic acid miR-31 inhibitor (as described above) comprises at least one (e.g. 1, 2, 3, 4, 5, 6, 7, 8, 9, 10, 12, 14, 16, 18 or 20) modified nucleotide(s). In one embodiment, the modified nucleotide is selected from: a locked nucleic acid (LNA) nucleotide, a 2′-O-methyl modified nucleotide, a 2′-O-methoxyethyl modified nucleotide, and a 2′-fluro modified nucleotide.

A nucleic acid miR-31 inhibitor of the invention may be constructed such that all of its nucleotides are modified nucleotides. Thus, in one embodiment, the nucleic acid miR-31 inhibitor consists of modified nucleotides. In one embodiment, said modified nucleotides consist of LNA nucleotides, or 2′-O-methyl modified nucleotides, or 2′-O-methoxyethyl modified nucleotides, or 2′-fluro modified nucleotides. In one embodiment, the nucleic acid miR-31 inhibitor is a synthetic 2′-O-methyl RNA oligonucleotide with a sequence complementary to SEQ ID NO: 2, and which competes with miR-31 target mRNAs with stronger binding to the miRNA-associated gene silencing complex.

The “percent sequence identity” between two or more nucleic acid sequences is a function of the number of identical positions shared by the sequences. Thus, % identity may be calculated as the number of identical nucleotides divided by the total number of nucleotides, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

Reference to nucleic acid(s) also embraces nucleic acid analogues. Nucleic acid analogues are composed of three parts: a phosphate backbone, a pucker-shaped pentose sugar, either ribose or deoxyribose, and one of four nucleobases. An analogue may have any of these altered. Typically the analogue nucleobases confer, among other things, different base pairing and base stacking proprieties. Examples include universal bases, which can pair with all four canon bases, and phosphate-sugar backbone analogues such as PNA, which affect the properties of the chain. Artificial nucleic acids include peptide nucleic acid (PNA), Morpholino and LNA, as well as glycol nucleic acid (GNA) and threose nucleic acid (TNA). Each of these is distinguished from naturally-occurring DNA or RNA by changes to the backbone of the molecule.

Thus, in one embodiment, the nucleic acid miR-31 inhibitor (as described above) is a nucleic acid analogue selected from: a peptide nucleic acid (PNA), a glycol nucleic acid (GNA), a threose nucleic acid (TNA), and a morpholino.

The nucleic acid miR-31 inhibitor (as described above) may comprise at least one (e.g. 1, 2, 3, 4, 5, 6, 7, 8, 9, 10, 12, 14, 16, 18 or 20) modified phosphodiester linkage. In one embodiment, the modified phosphodiester linkage is a phosphothiorate linkage. In one embodiment, all of the phosphodiester linkages are modified phosphodiester linkages.

The nucleic acid molecules of the invention may be made using any suitable process known in the art. Thus, the nucleic acid molecules may be made using chemical synthesis techniques. Alternatively, the nucleic acid molecules of the invention may be made using molecular biology techniques.

In one aspect, the invention provides a nucleic acid vector comprising a nucleic acid sequence encoding a nucleic acid miR-31 inhibitor as described above. In this scenario, the resultant nucleic acid may be produced in vivo.

The nucleic acid molecules of the present invention may be made by conventional expression of a nucleic acid vector encoding said nucleic acid molecule, followed by conventional nucleic acid recovery. Thus, in this scenario, the nucleic acid is produced in vitro. In one embodiment the nucleic acid vector is a plasmid.

The nucleic acid molecules of the present invention may be delivered into a target cell using a viral vector. The viral vector may be any virus which can serve as a viral vector. Suitable viruses are those which infect the target cells, can be propagated in vitro, and can be modified by recombinant nucleotide technology known in the art.

Thus, in one aspect, the invention provides a viral vector comprising a nucleic acid sequence encoding a nucleic acid inhibitor of miR-31 (as described above), for use in the prevention or treatment of atrial fibrillation in a subject.

Viral vectors suitable for use in the present invention include poxvirus vectors (such as non-replicating poxvirus vectors), adenovirus vectors, and adeno-associated virus (AAV) vectors (e.g. AAV type 9). Thus, in one embodiment, the viral vector is selected from an adenovirus vector, and adeno-associated virus vector, and a poxvirus vector.

As used herein, a non-replicating viral vector is a viral vector which lacks the ability to productively replicate following infection of a target cell. Thus, the ability of a non-replicating viral vector to produce copies of itself following infection of a target cell (such as a human target cell in an individual undergoing vaccination with a non-replicating viral vector) is highly reduced or absent. Such a viral vector may also be referred to as attenuated or replication-deficient. The cause can be loss/deletion of genes essential for replication in the target cell. Thus, a non-replicating viral vector cannot effectively produce copies of itself following infection of a target cell. Non-replicating viral vectors may therefore advantageously have an improved safety profile as compared to replication-competent viral vectors. A non-replicating viral vector may retain the ability to replicate in cells that are not target cells, allowing viral vector production. By way of example, a non-replicating viral vector (e.g. a non-replicating poxvirus vector) may lack the ability to productively replicate in a target cell such as a mammalian cell (e.g. a human cell), but retain the ability to replicate (and hence allow vector production) in an avian cell (e.g. a chick embryo fibroblast, or CEF, cell).

In one embodiment, the non-replicating poxvirus vector is selected from: a Modified Vaccinia virus Ankara (MVA) vector, a NYVAC vaccinia virus vector, a canarypox (ALVAC) vector, and a fowlpox (FPV) vector. MVA and NYVAC are both attenuated derivatives of vaccinia virus. Compared to vaccinia virus, MVA lacks approximately 26 of the approximately 200 open reading frames.

In one embodiment, the adenovirus vector is a non-replicating adenovirus vector (wherein non-replicating is defined as above). Adenoviruses can be rendered non-replicating by deletion of the E1 or both the E1 and E3 gene regions. Alternatively, an adenovirus may be rendered non-replicating by alteration of the E1 or of the E1 and E3 gene regions such that said gene regions are rendered non-functional. For example, a non-replicating adenovirus may lack a functional El region or may lack functional E1 and E3 gene regions. In this way the adenoviruses are rendered replication incompetent in most mammalian cell lines and do not replicate in immunised mammals. Most preferably, both E1 and E3 gene region deletions are present in the adenovirus, thus allowing a greater size of transgene to be inserted. This is particularly important to allow larger antigens to be expressed, or when multiple antigens are to be expressed in a single vector, or when a large promoter sequence, such as the CMV promoter, is used. Deletion of the E3 as well as the E1 region is particularly favoured for recombinant Ad5 vectors. Optionally, the E4 region can also be engineered.

In one embodiment, the adenovirus vector is selected from: a human adenovirus vector, a simian adenovirus vector, a group B adenovirus vector, a group C adenovirus vector, a group E adenovirus vector, an adenovirus 6 vector, a PanAd3 vector, an adenovirus C3 vector, a ChAdY25 vector, an AdC68 vector, and an Ad5 vector.

As discussed above, the present inventors have discovered that inhibition of miR-31 can be used to prevent or treat AF in a subject.

In one embodiment, administration of a nucleic acid miR-31 inhibitor (as described above) causes the subject to experience a reduction in one or more symptoms of AF. A non-exhaustive list of symptoms of AF includes: shortness of breath, reduced exercise capacity, and palpitations. The subject may also experience a reduction in the incidence of one or more complications of AF. A non-exhaustive list of complications of AF includes: embolic stroke, and heart failure.

In one embodiment, the subject experiences one or more of: a reduction in the incidence or recurrence of atrial fibrillation events, a reduction in the severity of an atrial fibrillation event, and a shortening in the duration of an atrial fibrillation event.

According to the invention, the nucleic acid miR-31 inhibitor is administered to the subject using any suitable method that enables the nucleic acid miR-31 inhibitor to reach the atrial myocardium.

The nucleic acid miR-31 inhibitor (as described above) may be administered to a subject by any suitable means known in the art. In one embodiment, the nucleic acid miR-31 inhibitor (as described above) is administered to the subject systemically (i.e. via systemic administration). Thus, in one embodiment, the nucleic acid miR-31 inhibitor (as described above) is administered to the subject such that it enters the circulatory system and is distributed throughout the body (including to the atrial myocardium). In another embodiment, the nucleic acid miR-31 inhibitor (as described above) is administered to the subject by local administration, for example by local administration to the heart (e.g. to the atrial myocardium) or to a tissue in the vicinity of the heart. Local administration may be carried out by any suitable means known in the art, for example by injection into or application on the atrial myocardium, or by injection into a peripheral vein or a coronary artery (thus transporting the nucleic acid miR-31 inhibitor to the atrial myocardium).

In another aspect, the invention provides a nucleic acid that inhibits miR-31, or a viral vector encoding a nucleic acid that inhibits miR-31, for use in a method of increasing nNOS protein levels in the atrial myocardium of a subject. In one embodiment, the nucleic acid is a nucleic acid miR-31 inhibitor as described above. In one embodiment, the viral vector is a viral vector as described above.

The amount of nucleic acid miR-31 inhibitor administered to a subject (i.e. dosage) may vary depending on the nucleic acid miR-31 inhibitor used. In one embodiment, a dosage may range from about 0.1 μg/kg to about 100 mg/kg, or from about 1 μg/kg to about 50 mg/kg, or from about 10 μg/kg to 5 mg/kg, of the subject's body weight.

The dosage of the nucleic acid miR-31 inhibitor may be determined by a medical professional, taking into account the severity of AF.

The nucleic acid miR-31 inhibitor may be administered to the subject in a single delivery, such as a bolus delivery. Alternatively, the nucleic acid miR-31 inhibitor may be administered to the subject using a continuous delivery technique, such as a timed infusion. The nucleic acid miR-31 inhibitor may be administered using a repeated delivery regimen, for example on an hourly, daily or weekly basis.

Nucleic acid miR-31 inhibitor dosages may be achieved by single or multiple administrations. By way of example, the nucleic acid miR-31 inhibitor may be administered to the subject in a regimen consisting of a single administration. Alternatively, the nucleic acid miR-31 inhibitor may be administered to the subject in a regimen comprising multiple administrations. For example, an administration regimen may comprise multiple administrations per day, or daily, weekly, bi-weekly, or monthly administrations. An example regimen comprises an initial administration followed by multiple, subsequent administrations at weekly or bi-weekly intervals. Another example regimen comprises an initial administration followed by multiple, subsequent administrations at monthly or bi-monthly intervals. Alternatively, administration of the nucleic acid miR-31 inhibitor can be guided by monitoring of AF symptoms in the subject. Thus, an example regimen comprises an initial administration followed by multiple, subsequent administrations carried out on an irregular basis as determined by monitoring AF symptoms in the subject.

Methods for delivering nucleic acids are known in the art and will be familiar to a skilled person. By way of example, suitable nucleic acid delivery methods include ionophoresis, microspheres (e.g. bioadhesive microspheres), nanoparticles, dendritic polymers, liposomes, hydrogels, cyclodextrins, and proteinaceous vectors.

For administration to a subject, the nucleic acid miR-31 inhibitor (or a viral vector encoding said nucleic acid miR-31 inhibitor) may be formulated as a pharmaceutical composition comprising a nucleic acid miR-31 inhibitor (as described above) or a viral vector (as described above) (as active ingredient). Such a pharmaceutical composition can be formulated according to known methods for preparing pharmaceutical compositions, such as by combining a nucleic acid miR-31 inhibitor or viral vector with a pharmaceutically acceptable carrier. Non-limiting examples of pharmaceutically acceptable carriers including water, saline and phosphate-buffered saline.

The pharmaceutical composition in addition to a pharmaceutically acceptable carrier can further be combined with one or more of a salt, excipient, diluent, albumin, immunoregulatory agent and/or antimicrobial compound. Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like. Suitable excipients are, for example, water, saline, dextrose, glycerol, ethanol, or the like and combinations thereof.

In addition, if desired, the pharmaceutical compositions may contain minor amounts of auxiliary substances such as wetting or emulsifying agents, and/or pH buffering agents. Examples of buffering agents include, but are not limited to, sodium succinate (pH 6.5), and phosphate buffered saline (PBS; pH 6.5 and 7.5).

Thus, in one aspect, the invention provides a pharmaceutical composition comprising a nucleic acid miR-31 inhibitor (as described above) or a viral vector (as described above) and a pharmaceutically acceptable carrier. The pharmaceutical composition may contain 5% to 95% of nucleic acid miR-31 inhibitor or viral vector, such as at least 10%, at least 25%, at least 40%, or at least 50, 55, 60, 70 or 75%.

In one aspect, the invention provides a method for diagnosing or predicting the risk of AF in a subject, said method comprising:

(i) determining the amount of miR-31 in a sample from the subject;

(ii) comparing the amount of miR-31 in the sample with a reference standard; and

(iii) identifying a difference in the amount of miR-31 in the sample relative to the reference standard;
wherein an increase in the amount of miR-31 in the sample compared to the reference standard correlates with the presence of, or an increased risk of, atrial fibrillation in the subject; and
wherein a decrease, or no difference in the amount of miR-31 in the sample compared to the reference standard correlates with the absence of an increased risk of AF in the subject.

Advantageously, the method of the invention enables the identification of subjects having or at an increased risk of AF (as compared to the general population), enabling the commencement of appropriate treatment or prophylaxis. By way of example, a subject identified by the method of the invention as being at increased risk of AF may be administered a nucleic acid miR-31 inhibitor as described above or a viral vector as described above.

The sample may be an isolated sample from the subject of any suitable tissue. The sample may be a blood sample, a plasma sample, a serum sample, or an atrial tissue sample.

The amount of miR-31 in the sample may be determined by any suitable method known in the art. In the present context, the amount of miR-31 refers to the level or quantity of miR-31, for example, units mass RNA per units volume sample. The amount of miR-31 may also be determined by measuring relative values, for example as measured using qRTPCR (e.g. Ct values per 25 ng or recalculated RNA concentration loaded per experiment). Techniques for measuring mRNA concentration are well known in the art, and include reverse transcriptase polymerase chain reaction (RTPCR) and quantitative RTPCR (qRTPCR).

The reference standard acts as a control and enables the comparison of the amount of miR-31 in the sample from the subject (which is the test sample, from the test subject) with the amount of miR-31 in a reference sample.

In one embodiment, a reference standard comprises (or consists of) a sample from a reference subject or subjects, wherein the reference subject is a subject other than the test subject, and wherein the reference subject does not have and has not experienced atrial fibrillation.

In one embodiment, a reference standard comprises (or consists of) a set of data relating to the amount of miR-31 in a reference sample or samples derived from a reference subject or subjects, wherein the reference subject is a subject other than the test subject, and wherein the reference subject does not have and has not experienced atrial fibrillation. The set of data is derived by measuring the amount of miR-31 in the reference sample or samples. Said measuring may be carried out using any suitable technique known in the art.

The reference standard may be matched to the test sample. Thus, in one embodiment, when the sample is an atrial tissue sample (e.g. an atrial myocardium sample), the reference standard relates to the amount of miR-31 in atrial tissue.

In one embodiment, an increase in the amount of miR-31 in the sample compared to the test sample of at least 1.5-fold (e.g. 1.6, 1.7, 1.8, 1.9, 2.0, 2.1, 2.2, 2.3, 2.4, or 2.5-fold) correlates with the presence of, or an increased risk of, atrial fibrillation in the subject.

In one embodiment, an increase in the amount of miR-31 in the sample compared to the test sample of approximately 2-fold correlates with the presence of, or an increased risk of, atrial fibrillation in the subject.

The method of the invention may be used to monitor miR-31 levels in a subject over a period of time, for example following an AF episode. Thus, in one embodiment, wherein the subject has experienced an AF episode, the method further comprises one or more repetitions (e.g. 1, 2, 3, 4 or 5) of steps (i) to (iii) using at least one (e.g. 1, 2, 3, 4 or 5) further sample (or samples) that has (or have) been taken from the subject in a serial fashion over a period of time following the AF episode. Thus, the level of miR-31 in each sample may be determined to provide data on miR-31 levels over a period of time. In one embodiment, steps (i) to (iii) are repeated using samples that have been taken from a subject at intervals of 1, 2, 6, 12 or 24 hours; or 1, 2, 7, 14 or 28 days; or 1, 2, 3, 6 or 12 months. In one embodiment, the method of the invention is repeated using samples that have been taken from a subject at intervals of 1, 2, 6, 12 or 24 hours; or 1, 2, 7, 14 or 28 days; or 1, 2, 3, 6 or 12 months; following the AF episode.

The amount of miR-31 determined to be in a first sample from the subject may subsequently form the basis of a reference standard, against which the amount of miR-31 in subsequent samples that have been taken from the same subject may be compared. Thus, in one embodiment, the reference standard is derived from a first sample that has been taken from the subject at a first time point; and step (i) is carried out on a second sample that has been taken from the subject subsequent to the first time point. Said embodiment may further comprise the steps of determining the amount of miR-31 in further samples (e.g. 1, 2, 3, 4, 5, 6 or 10 further samples) that have been taken from the subject in a serial fashion over a period of time, and comparing the amount of miR-31 in each further sample with the amount of miR-31 in the reference sample. By way of example, the second and further samples may be samples that have been taken from the subject at intervals of 1, 2, 6, 12 or 24 hours; or 1, 2, 7, 14 or 28 days; or 1, 2, 3, 6 or 12 months; following the first time point.

### LIST OF SEQUENCES

All nucleotide sequences are written in the direction 5′-3′.

SEQ ID NO: 1. Nucleic acid sequence complementary to miR-31, with nucleotides complementary to nucleotides 2-7 of miR-31 underlined:

AGCUAUGCCAGCAUCUUGCCU

SEQ ID NO: 2. Nucleic acid sequence of miR-31, with nucleotides 2-7 of the seed region underlined:

AGGCAAGAUGCUGGCAUAGCU

SEQ ID NO: 3. miR-31 precursor sequence, with sequence of mature miR-31 underlined:

miR-31 precurser sequence, with sequence of
mature miR-31 underlined:
SEQ ID NO: 3
GGAGAGGAGGCAAGAUGCUGGCAUAGCUGUUGAACUGGGAACCUGCUA
UGCCAACAUAUUGCCAUCUUUCC.
SEQ ID NO: 4
CUUGCC.
SEQ ID NO: 5
GGCAAG
SEQ ID NO: 6
AGGCAAGATGCTGGCATAGCT
SEQ ID NO: 7
TCCGTTCTACGACCGTATCGA
SEQ ID NO: 8
TGGCAAGAATGAAAAGGC
SEQ ID NO: 9
GGCAAGTTATTTAGCTAT

### LIST OF FIGURES

FIG. 1. Short- and long-term AF cause a bi-atrial reduction in NOS activity due to loss of myocardial nNOS.

Atrial NOS activity (measured by HPLC) is significantly supressed in human right atrial (RA) homogenates (n=17-19) (a), and in goat RA and left atrial (LA) tissue (n=8-13) in both short and long-term AF (b). nNOS protein is profoundly reduced in goat RA and LA tissue after 2 weeks (2W-AF) and 6 months of atrial fibrillation (6M-AF); n=12-43 (c, d). Reduction in nNOS protein was also observed in isolated human RA myocytes from patients with AF compared to patients in sinus rhythm (SR, n=8). Murine brain homogenate was used as a positive control for nNOS (e). Immunolocalisation experiments confirmed a reduction in myocardial nNOS (green) in human RA tissue cryosections (scale bar—50 μm; DAPI—blue, f). †P<0.05 between RA and LA; *P<0.05, **P<0.01, ***P<0.001—vs controls in SR.

FIG. 2. nNOS inhibition or gene deletion decreases action potential duration (APD) and abolishes the rate-adaptation of APD in human atrial myocytes, and increases AF inducibility in mice in vivo.

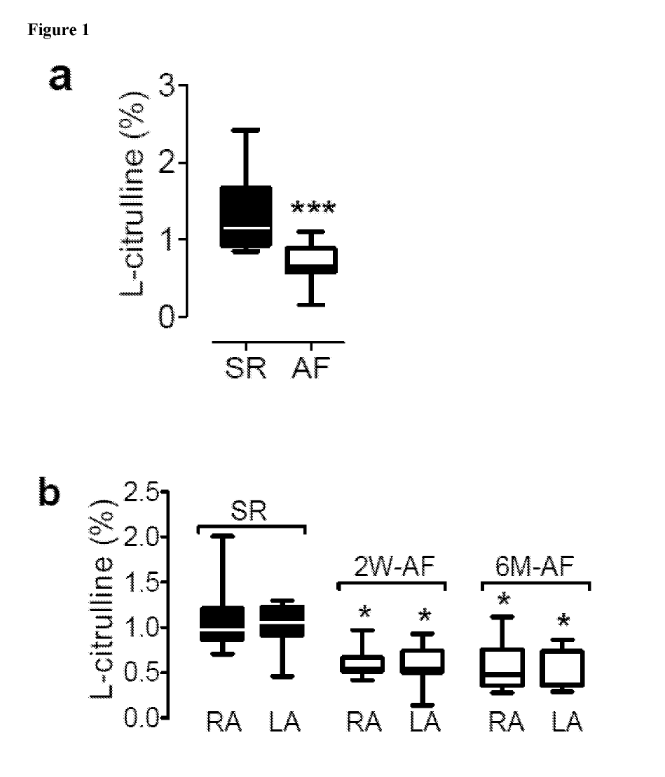

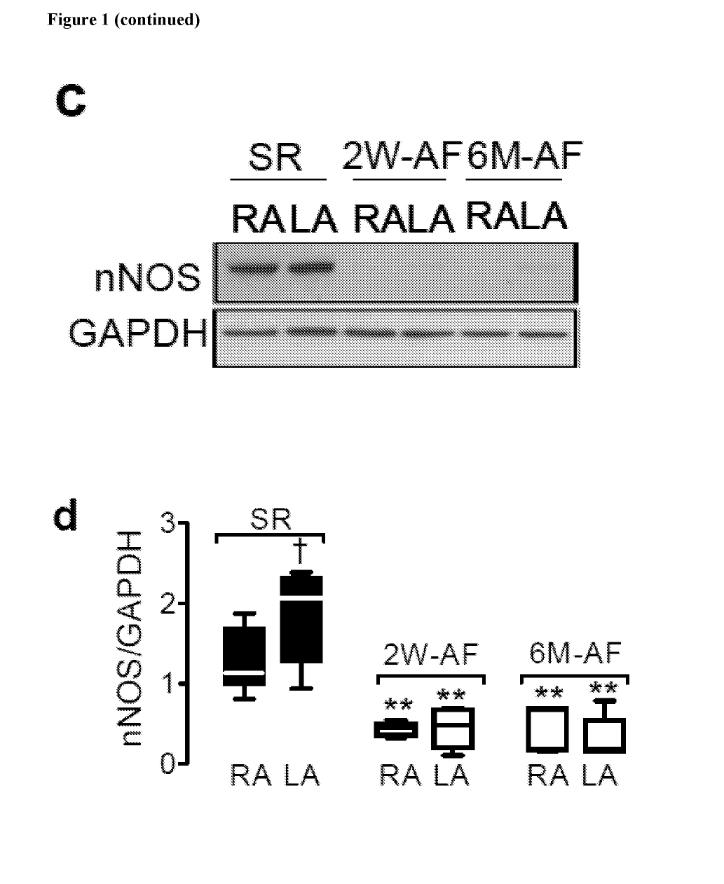

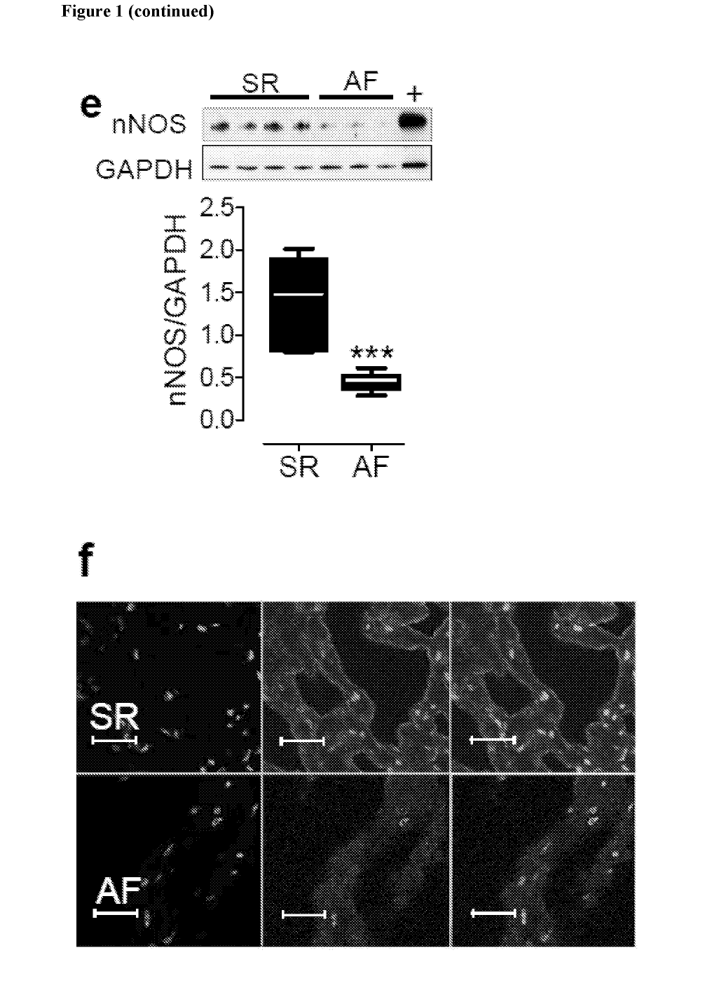

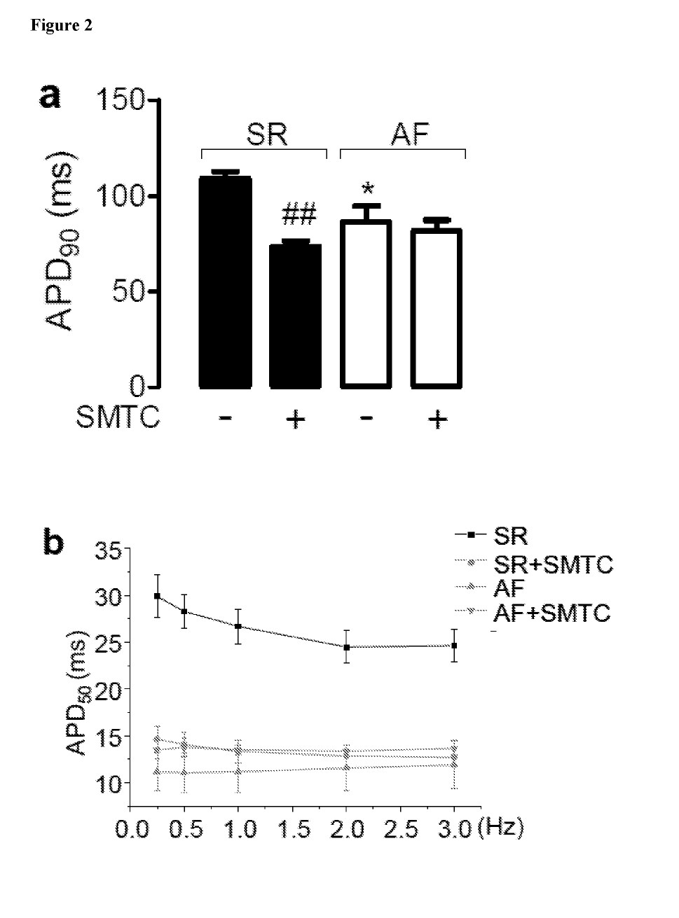

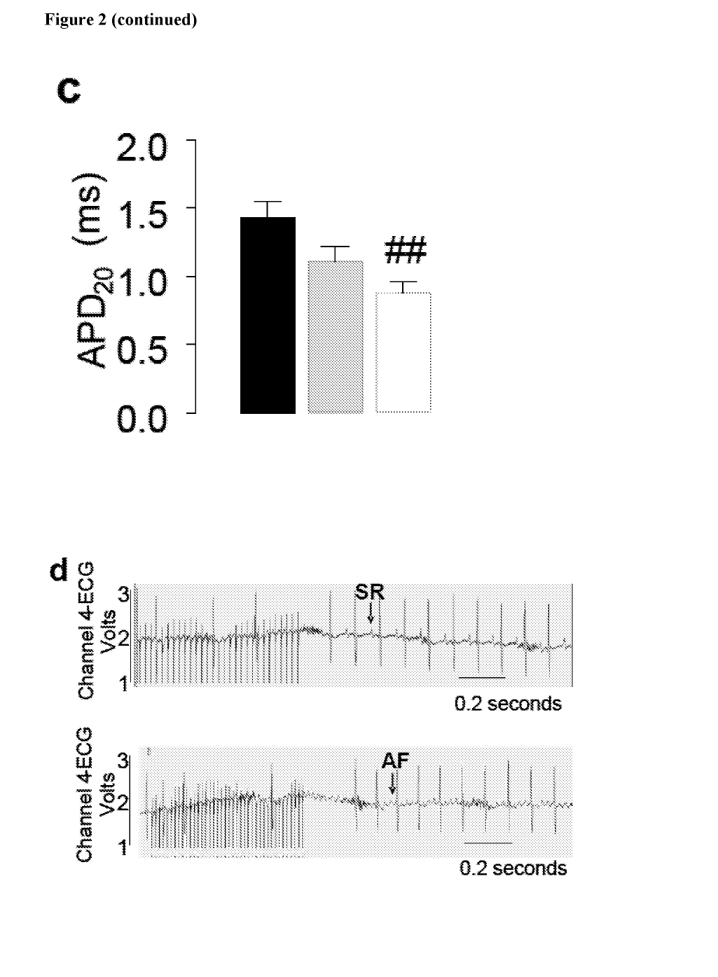

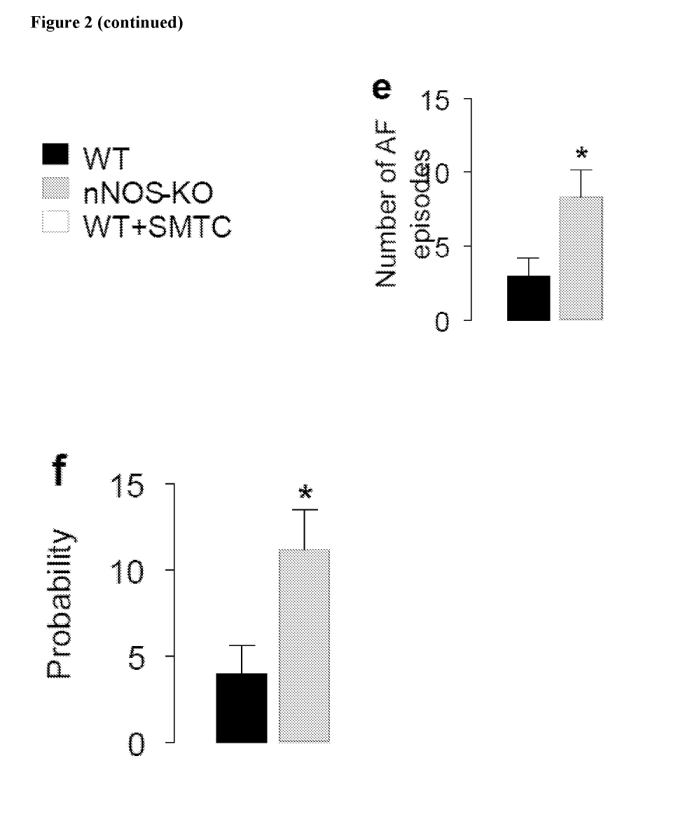

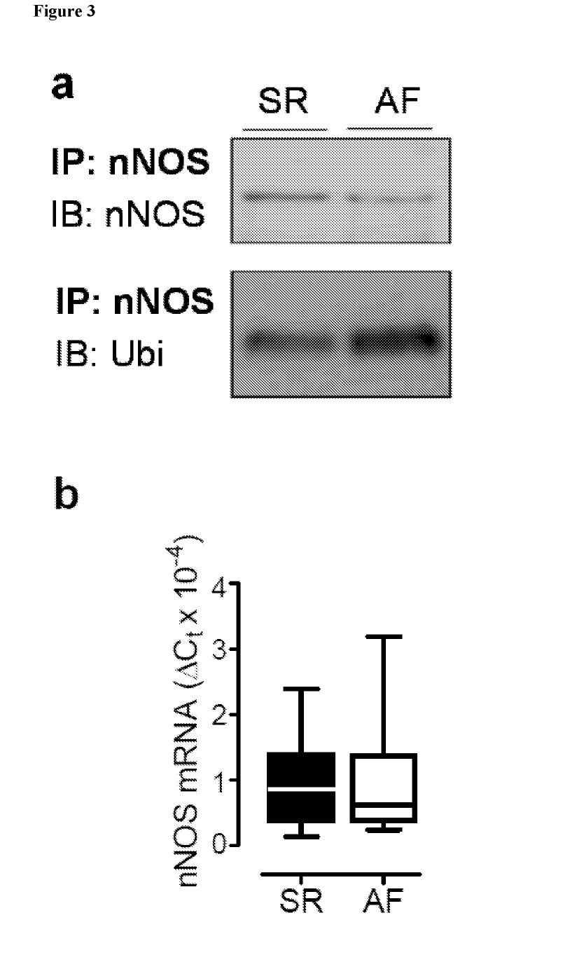

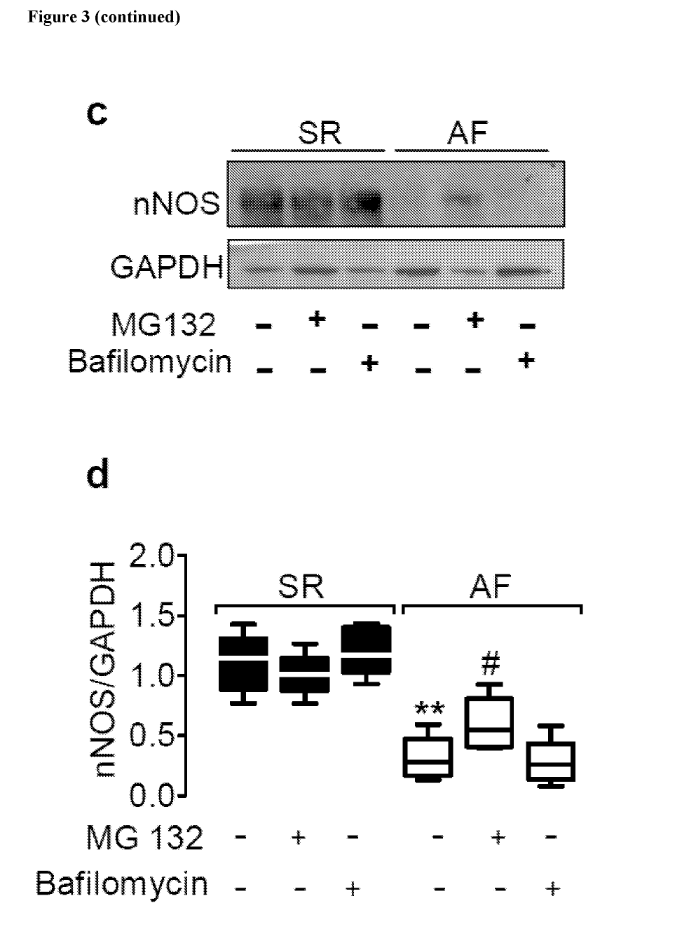

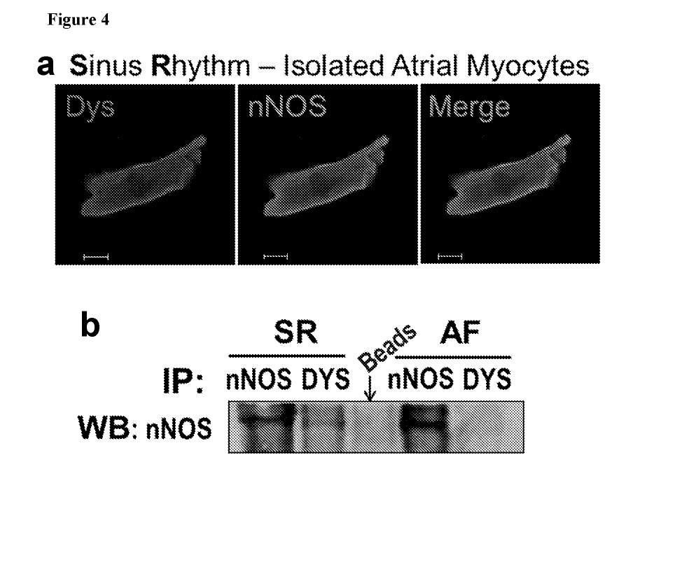

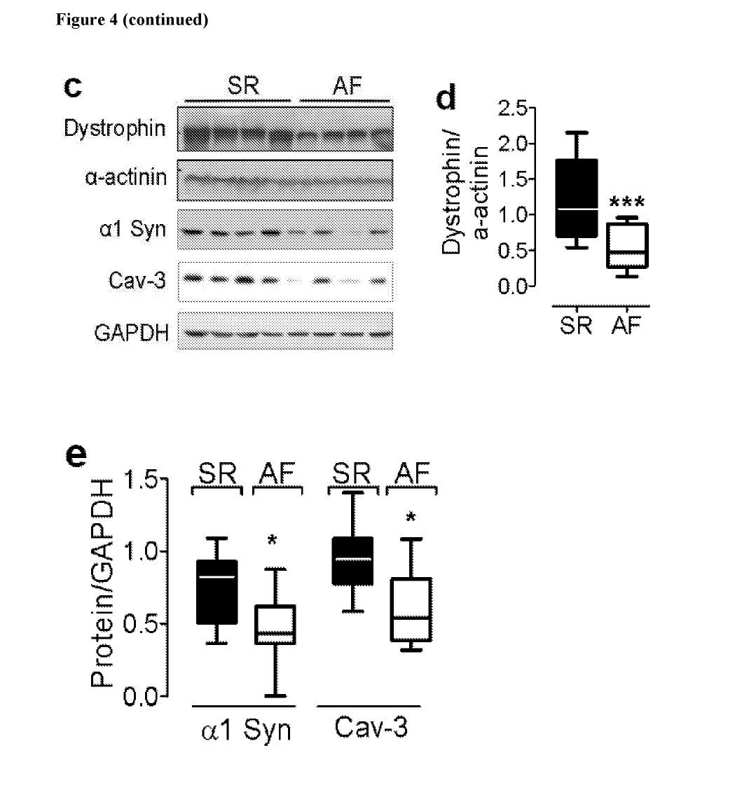

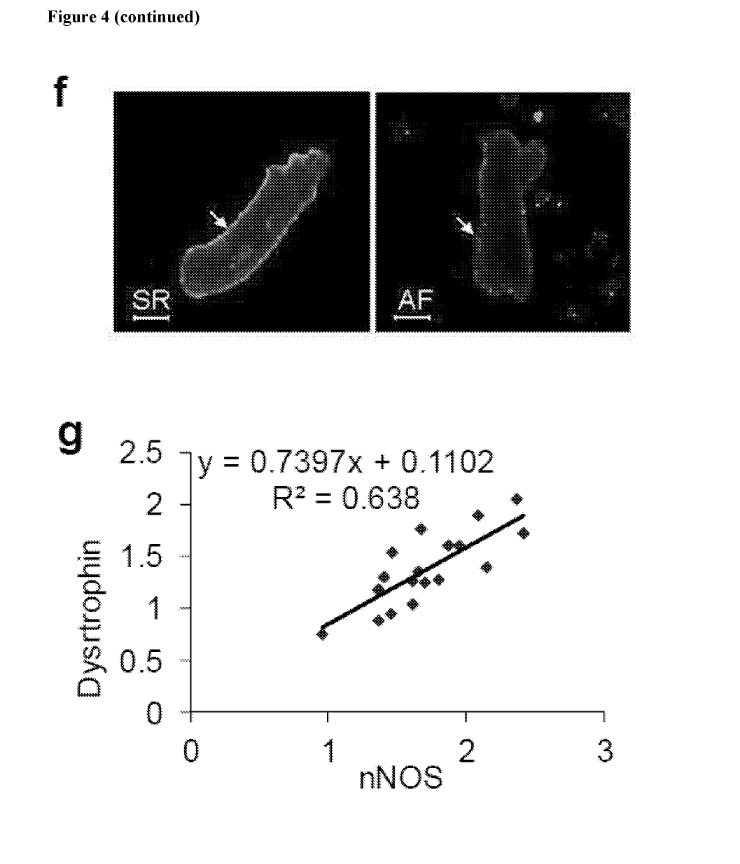

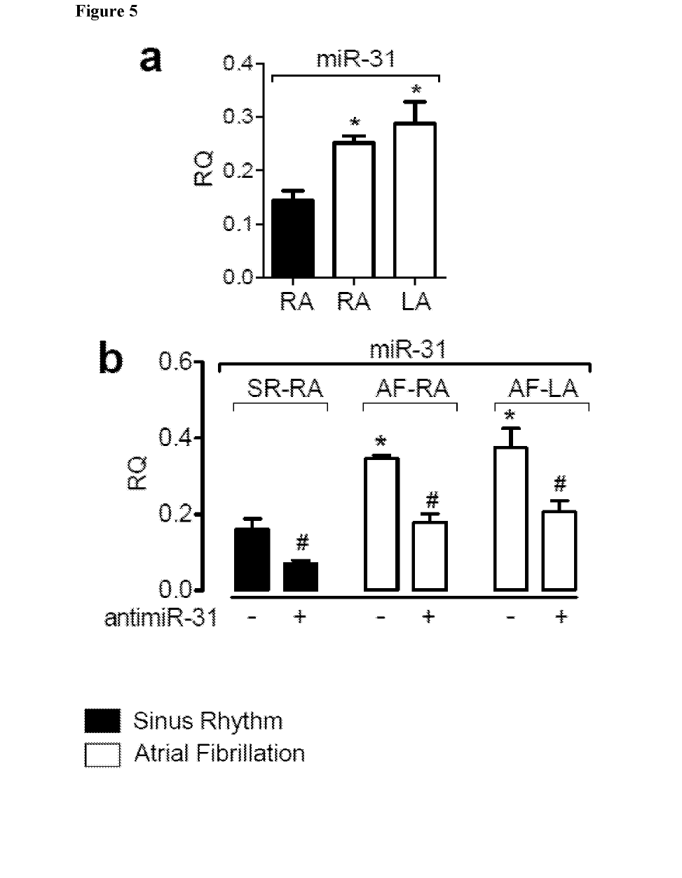

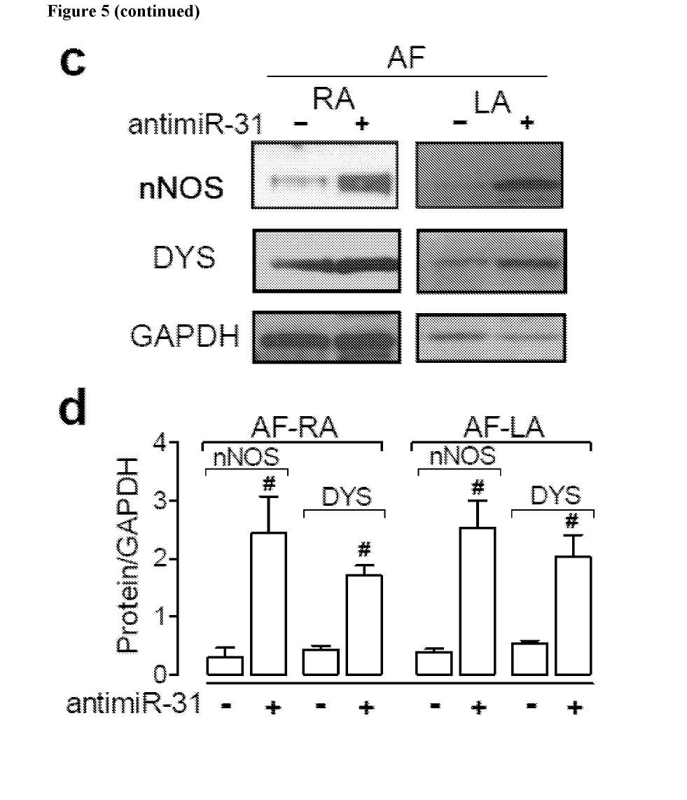

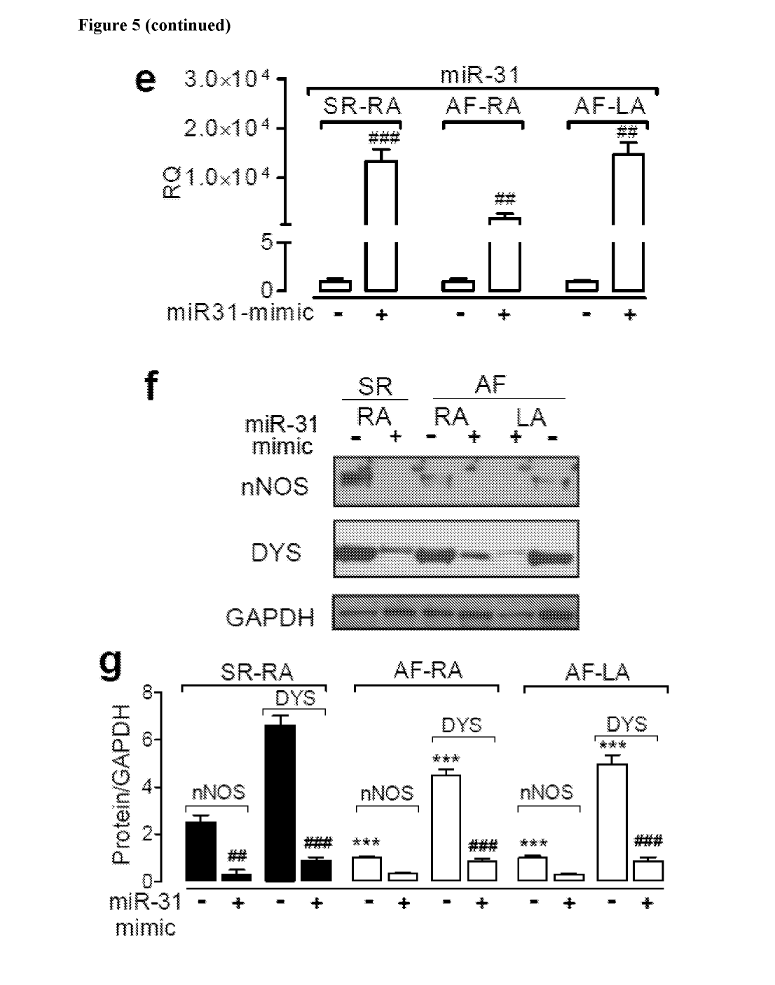

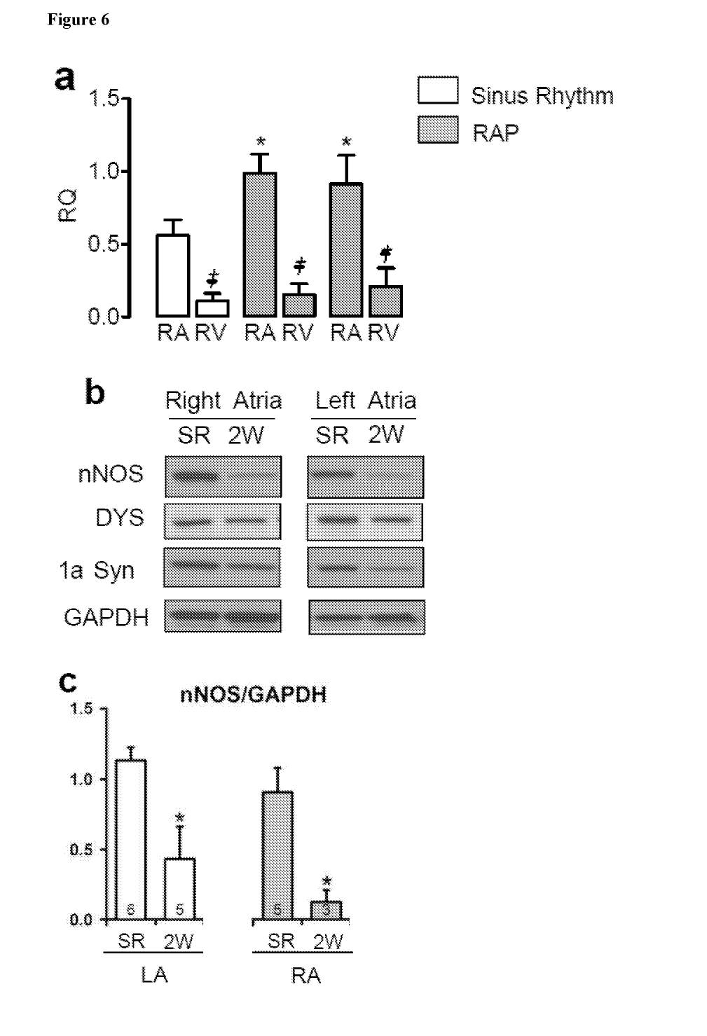

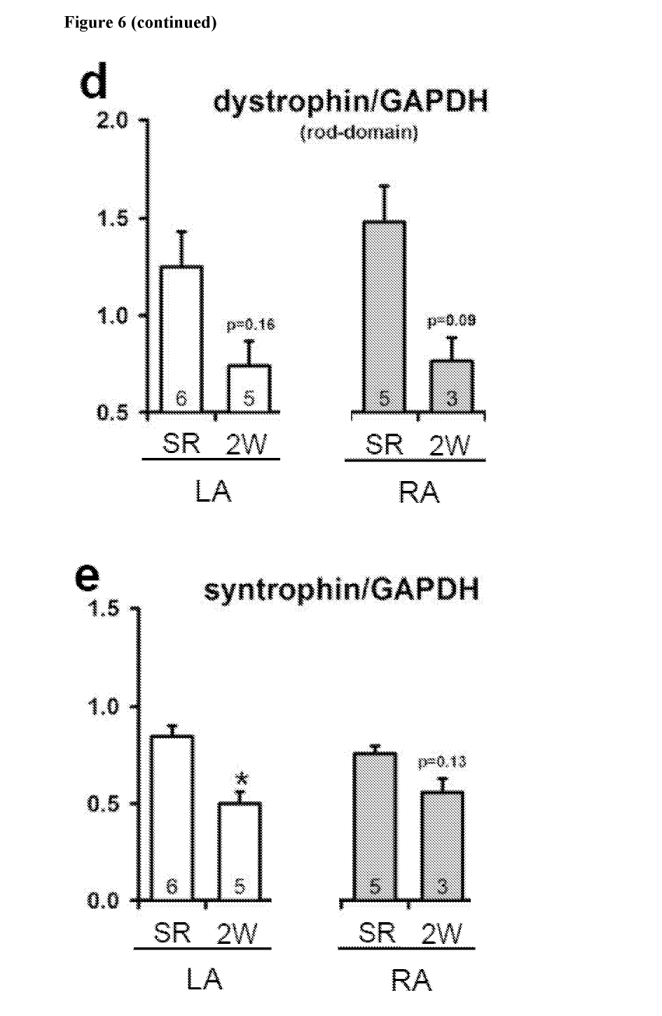

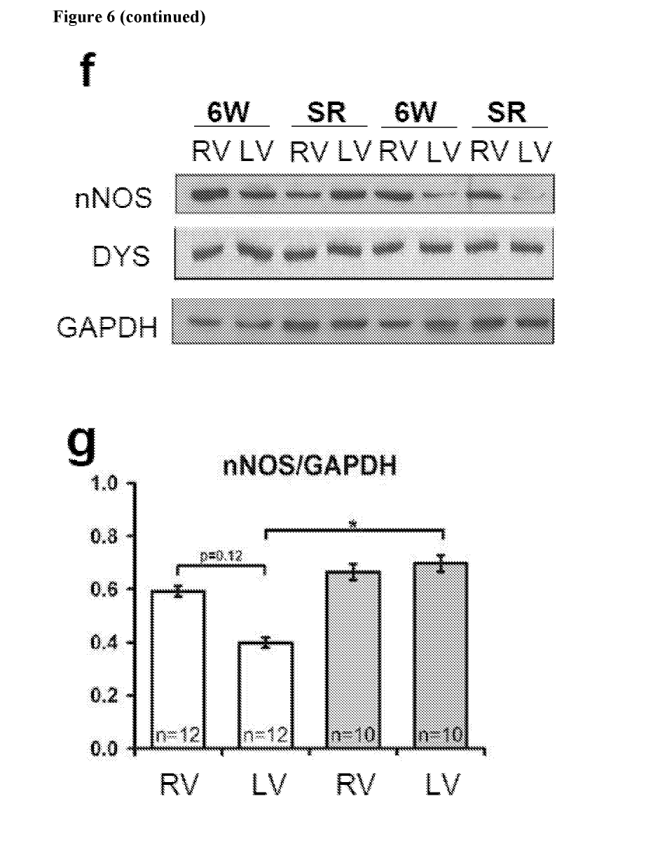

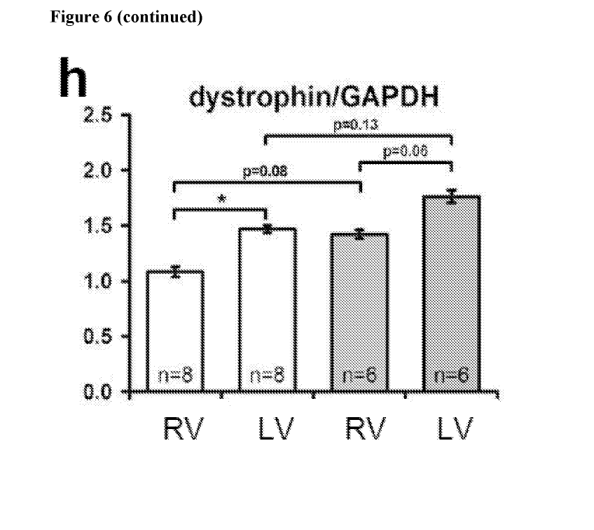

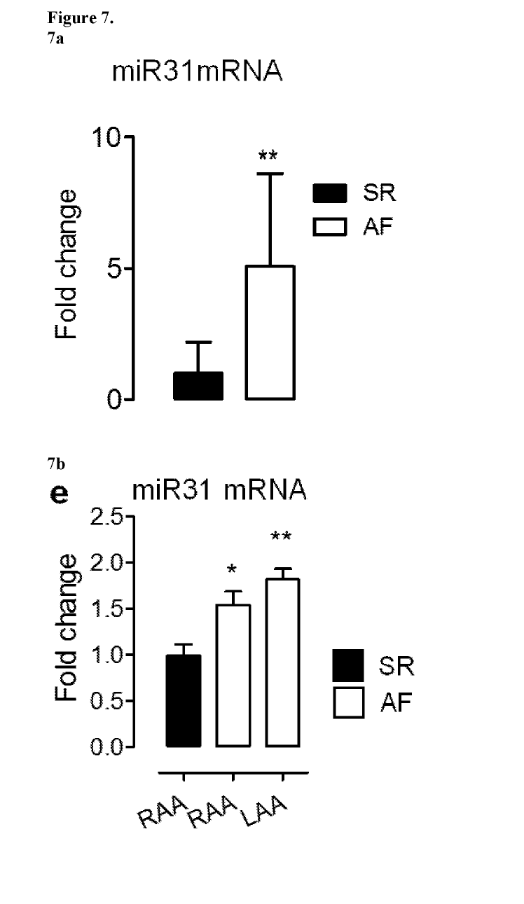

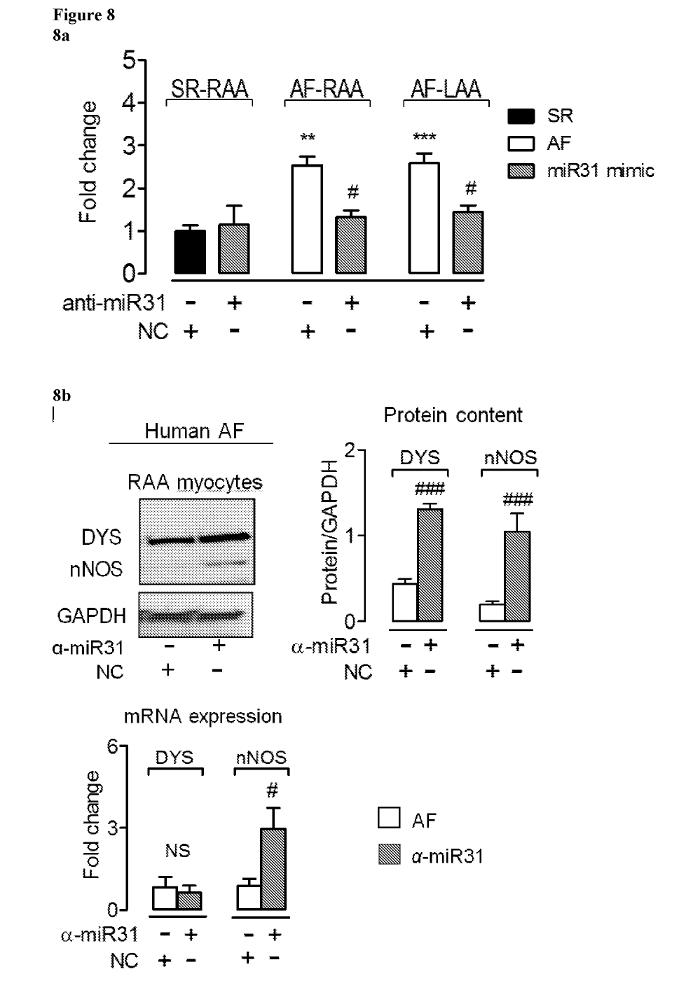

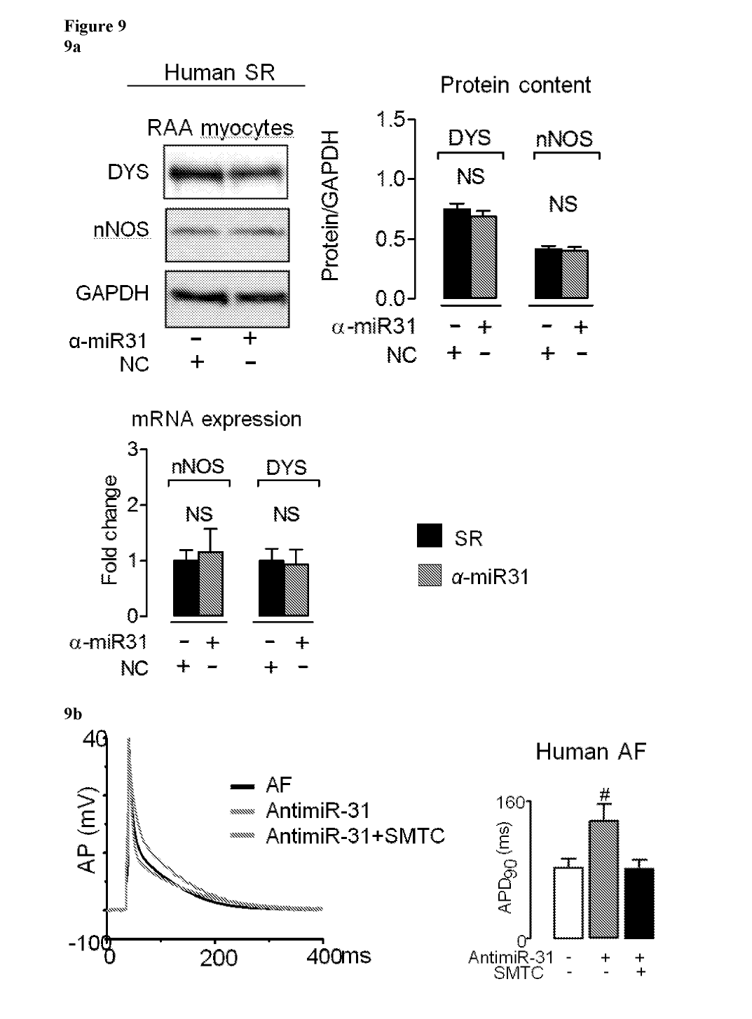

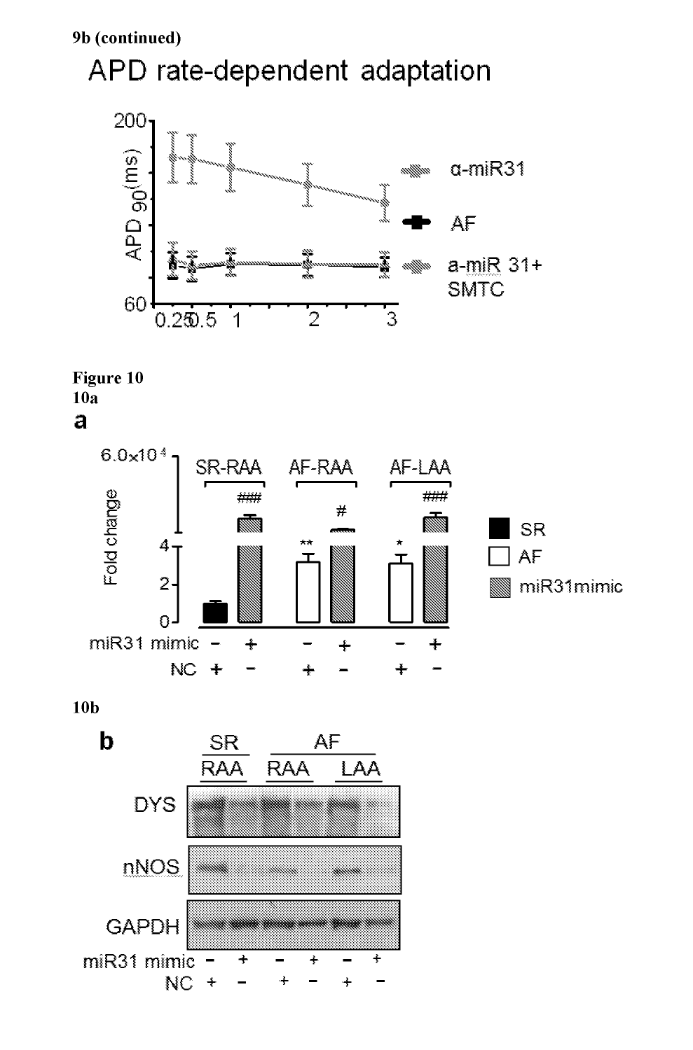

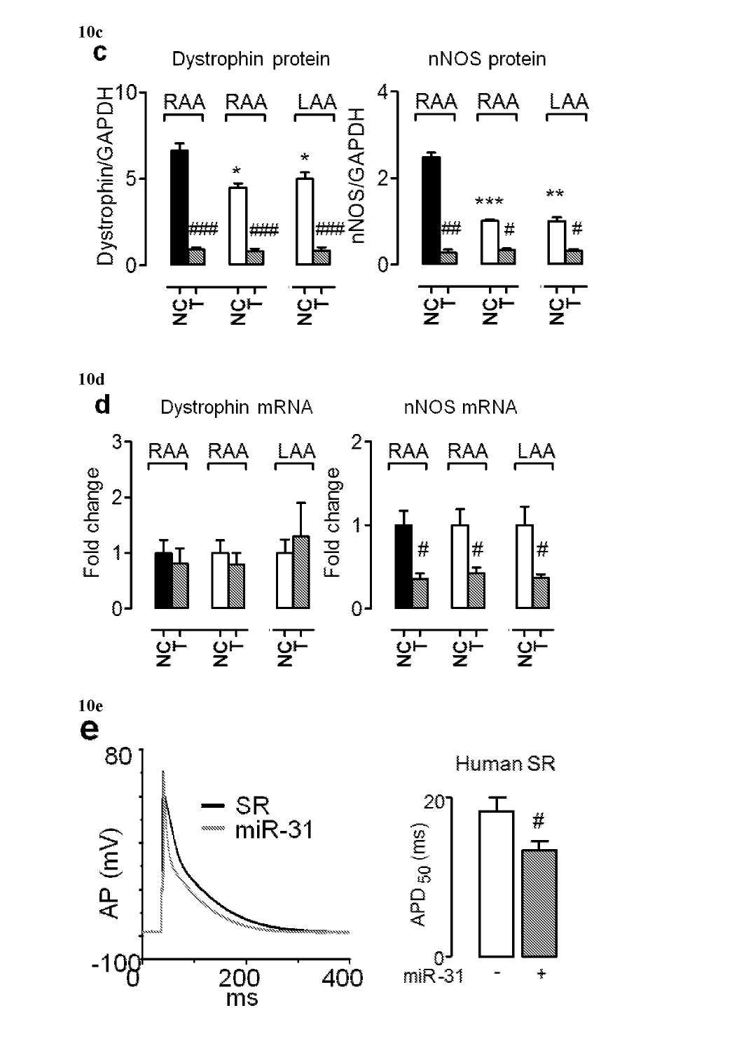

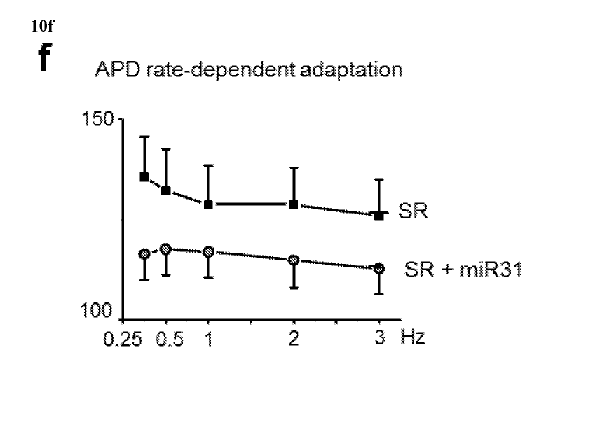

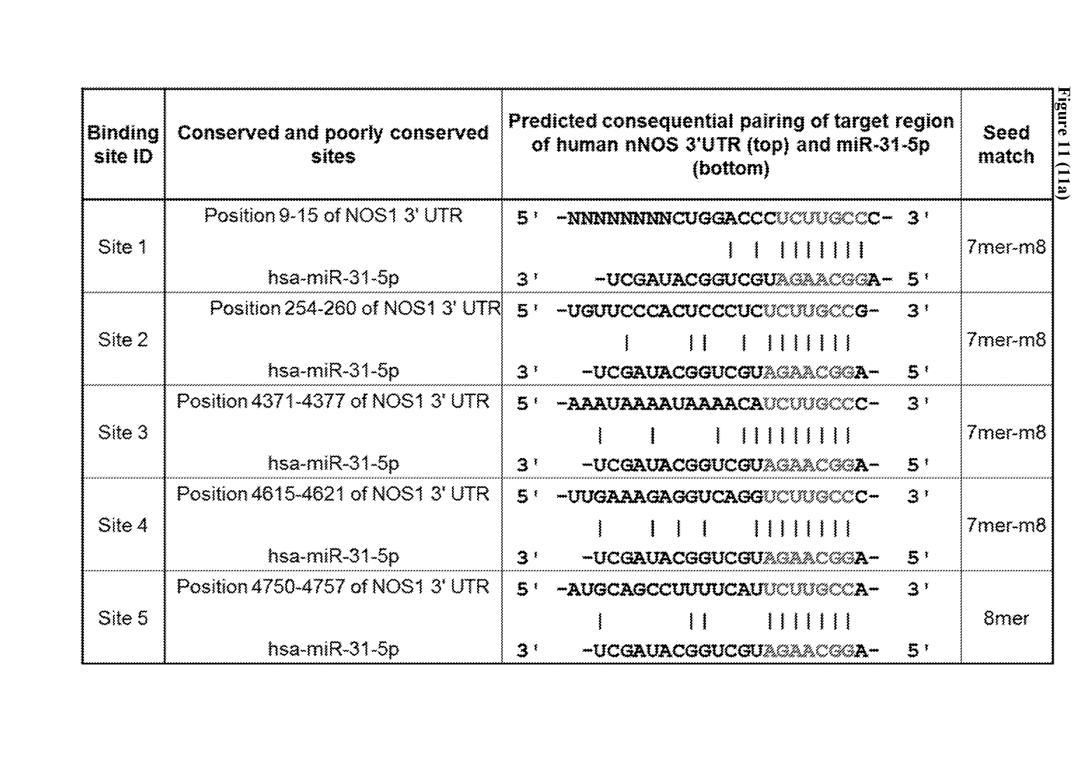

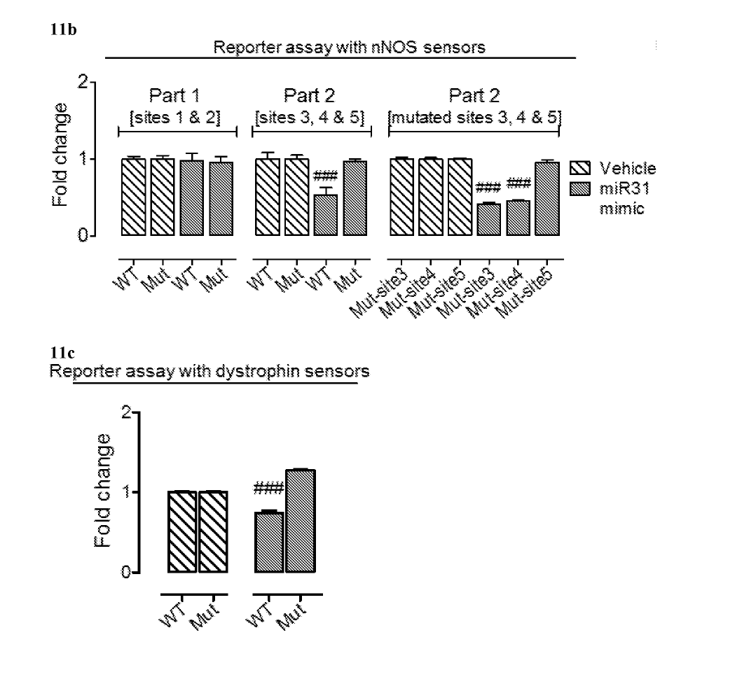

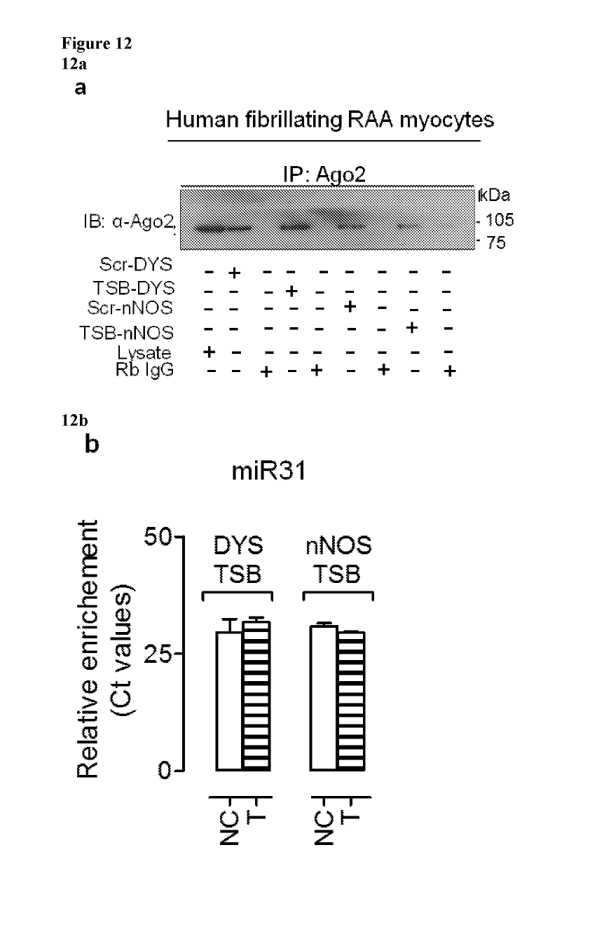

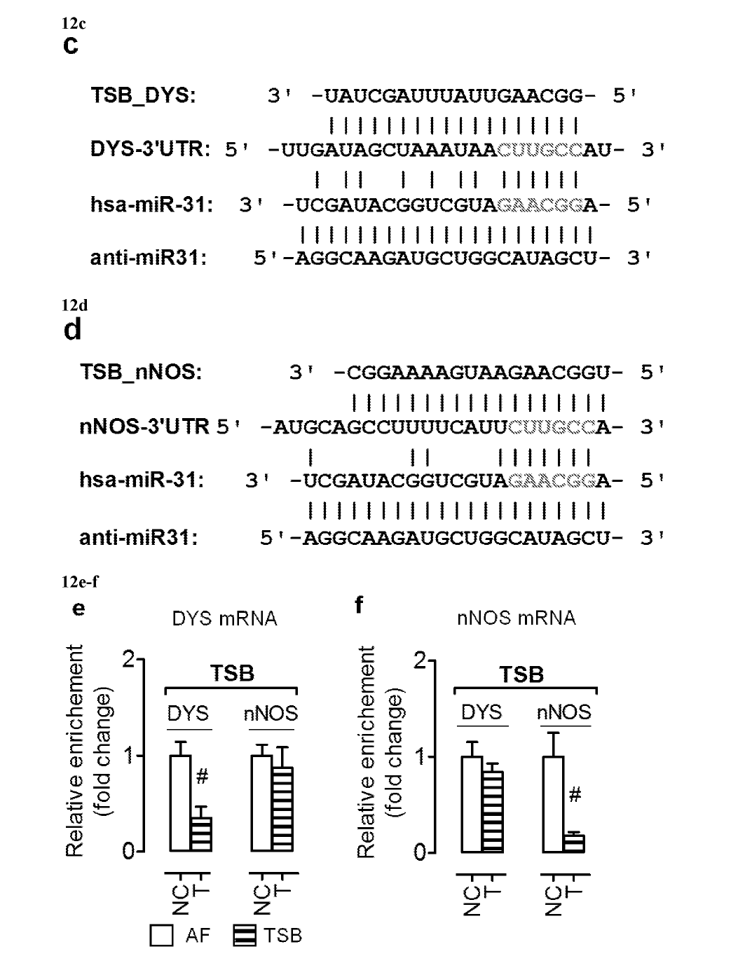

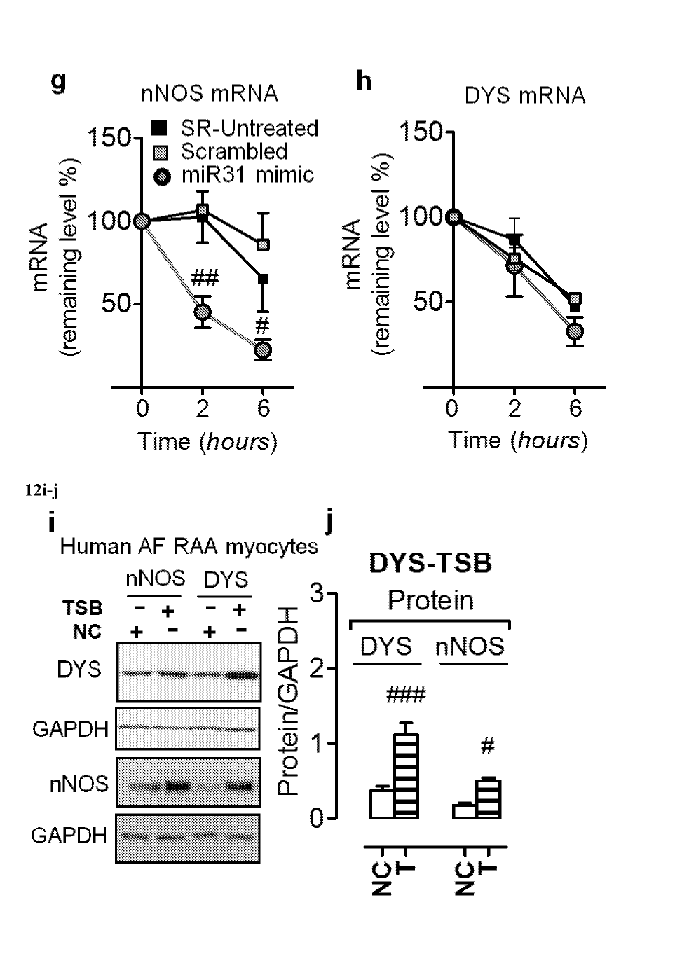

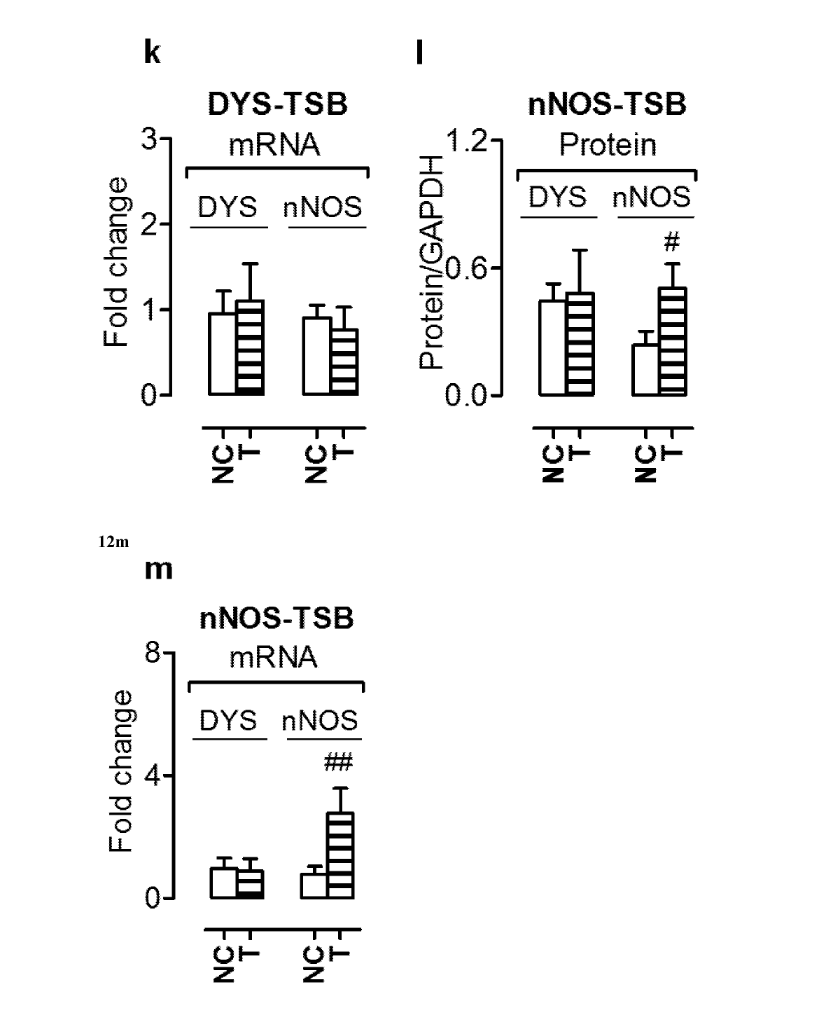

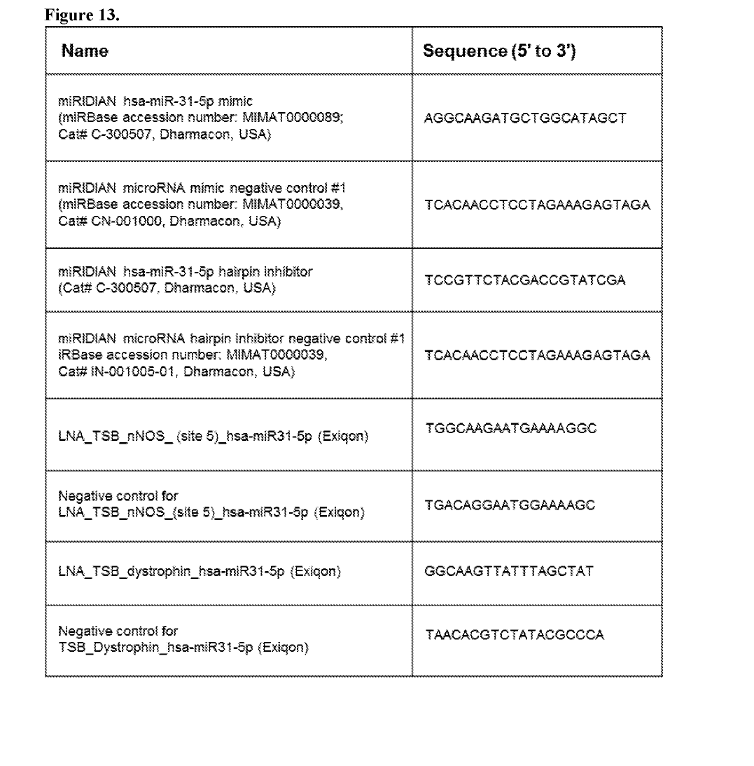

APD90 is shorter in RA myocytes from patients with AF vs. SR controls; nNOS inhibition with SMTC (100 nM) shortens APD and abolishes the APD rate-adaptation in atrial myocytes from patients in SR (n=31), but not in AF (n=11) (a, b). These findings are recapitulated in murine RA myocytes in the presence of nNOS inhibition (n=35) or gene deletion (nNOSKO, n=11) (c). nNOS gene deletion significantly increases the inducibility of AF in response to trans-oesophageal burst stimulation (d-f). n=15 mice/genotype. *P<0.05—vs controls in SR or between genotypes, ##P<0.01—vs non-treated. All abbreviations are as defined in FIG. 1.

FIG. 3. In AF, lower nNOS abundance is due to increased nNOS degradation by the ubiquitin-proteosomal system.

In the presence of AF, ubiquitination of nNOS protein is increased in human atrial tissue as shown by immunoprecipitation. Protein load of AF samples was increased to match the nNOS content to that seen in SR; n=6 (a). Gene expression of nNOS (quantitative RT-PCR) in human right atrial (RA) tissue is unaltered by AF, n=22-30 (b), whereas, inhibition of the proteosomal system with MG132 (but not of autophagy with bafilomycin A1) partially restores nNOS protein (n=4-5) (c, d). **P<0.01 vs controls in SR, #P<0.05 vs non-treated. All abbreviations are as defined in FIG. 1.

FIG. 4. AF causes a reduction in dystrophin and dystrophin-associated proteins.

Immunolocalization and co-immunoprecipitation show that nNOS is partially co-localised with dystrophin in patients in SR, but not in AF (a, b). Immunoblots show a lower content of dystrophin, caveolin-3 (Cav-3) and α1 syntrophin (aα1 Syn) in RA homogenates from patients with AF, n=17-19 (c-e). Immunostaining of human RA myocytes shows that dystrophin (green) is reduced and patchy (pointed by arrows) in AF compared with SR; (scale bar—25 um) (f). The RA protein level of nNOS is strongly correlated that of dystrophin (R2—0.64) (g). *P<0.05, ***P<0.001—vs controls in SR. All abbreviations are as defined in FIG. 1.

FIG. 5. In patients with AF, expression of miR-31 is upregulated in both RA and LA myocytes. Inhibition (but not stimulation) of miR-31 recovers myocardial dystrophin and nNOS.

miR-31 expression is upregulated in RA and LA myocytes from patients with AF; n=18-19/group (a). Transfection of isolated human RA and LA myocytes with anti-miR-31 (20 nM, 48-72 hours) decreased miR-31 expression both in SR and in AF (b), and restored protein level of both dystrophin and nNOS in the atrial myocytes from patients with AF (n=3), (c, d). By contrast, upregulation of miR-31 with a mimic (20 nM, 48-72 hours) (e) dramatically reduced dystrophin and nNOS content in human RA and LA myocytes from both SR and AF patients, n=3 (f, g). *P<0.05, ***P<0.001—vs controls in SR, #P<0.05##P<0.01, ###P<0.001—vs non-treated. RQ—relative quantification, DYS—dystrophin; all other abbreviations are as defined in FIG. 1.

FIG. 6. miR-31 is predominantly expressed in the atria. Rapid atrial pacing (RAP) in pigs greatly augments miR-31 expression in the atria but not in the ventricles.

In control pigs in SR miR-31 expression (qRT-PCR) is greater than in the RA than in the right ventricle (RV), and is selectively upregulated in the atria by rapid atrial tachypacing (RAP); n=4/group (a). In the same model, RA and LA tissue level of nNOS, dystrophin (DYS) and al syntrophin (al Syn) protein is reduced after 2 weeks (2 W) or 6 weeks (6 W) of RAP (b-e). By contrast, nNOS content is preserved in the RV and is upregulated in the left (LV) in response to 6 W RAP (f, g). Dystrophin content is slightly higher in the LV tissue vs RV, and is not affected by RAP in either ventricle (f, h). †P<0.05, P<0.001 between RV and LV; *P<0.05—vs controls in SR. RQ—relative quantification.

FIG. 7.

7a. In AF (n=19 patients), miR31 is upregulated in RA homogenates compared to the control patients in SR (n=17); **P<0.01 using non-parametric Mann Whitney test.

7b. In the presence of AF, the level of miR31 is significantly increased in human RA and LA myocytes and atrial homogenates from patients vs. SR (n=14 vs. n=11 patients; *P<0.05 **P<0.01 vs controls in SR, Mann Whitney test).

FIG. 8.

8a. Transient transfection (72 hours) of isolated human RAA (n=5) and LAA (n=5) myocytes with 20 nM miRIDIAN miR31 hairpin inhibitor (anti-miR31, Dharmacon) reduces expression of miR31 (red bars) to the level observed in control myocytes isolated from patients in SR (n=5/group, black bar), whereas, anti-miR31 has no effect on miR31 expression in SR. **P<0.01, ***P<0.001 vs controls in SR, #P<0.05 vs negative control (NC) using 1-way ANOVA for repeated measurements with Bonferroni correction. Data are expressed as fold change to the controls in SR.

8b. Transient transfection of RA myocytes with a-miR-31 recovers both DYS and nNOS protein content (representative immunoblots and mean data, n=5 patients per group); ###P<0.001 vs negative control (NC, 20 nM, Dharmacon), 1-way ANOVA with Bonferroni correction. a-miR31 did not affect DYS mRNA (n=11), but significantly increased nNOS gene expression (n=10); #P<0.05 vs NC, 1-way ANOVA with Bonferroni correction.

FIG. 9.

9a. In the presence of SR transfection of RA myocytes (6 patients) with a-miR31 does not alter DYS and nNOS protein content (i) and gene expression (j); P=1, 1-way ANOVA with Bonferroni correction.

9b. Inhibition of Iₜₒ and Iₖᵤᵣ using 4-AP prolongs APD in human RA myocytes and restores the APD rate dependent adaptation. Representative AP traces in RA myocytes from patients in SR exposed to 2 doses of 4-AP in the presence or absence of nNOS inhibition with SMTC.

9c. Average data show that both doses of 4-AP (100 red line and 500 blue line) prolong APD and abolish the difference in APD induced by SMTC.

FIG. 10.

Transient transfection of human RAA myocytes with miR31 mimic increases mir31 levels (a, n=7 in SR-RAA and n=6 in all AF groups) reduces dystrophin and nNOS protein (b, representative immunoblots and (c) mean data, n=4 patients in each group) and (e) nNOS gene expression in patients in SR (n=10) and in patients with AF (n=12). By contrast, treatment with miR31 mimic has no effect on the DYS mRNA in either SR (n=12) or AF (n=10). *P<0.05, **P<0.01 ***P<0.001 vs controls in SR and #P<0.05, ##P<0.001, ###P<0.001 vs negative control (NC) using 1-way ANOVA with Bonferroni correction. Dystrophin and nNOS gene expression were normalized to GAPDH.

FIG. 11.

11a. Predicted conserved (site 1) and poorly conserved (sites 2, 3, 4 and 5) miR31-5p binding sites on the human nNOS 3′UTR.

11b. HEK293 cells were co-transfected (48 hours) with reporter constructs containing 2 fragments (˜500 bp) of the nNOS 3′UTR region containing the mir31 binding putative sites (Part1, site 1 & 2 and Part2, sites 3, 4 & 5) and a miR31 mimic (10 nM, miRIDIAN, Dharmacon). A reduction in miR31-mediated GFP fluorescence observed in the wild type (WT) Part 2 construct was abolished in the mutated (Mut) constructs lacking miR31 binding site. Further analysis of Part 2, identified site 5 as the functional mir31 site on the nNOS 3′ UTR.

11c. HEK293 cells were co-transfected (48 hours) with reporter constructs containing the DYS 3′UTR region containing the mir31 binding putative site. A reduction in miR31-mediated GFP fluorescence observed in the wild type (WT) is abolished in the mutated (Mut) constructs lacking miR31 binding site. Reporter assays were performed in triplicates and are representative of at least 3 independent experiments. ###P<0.001 vs vehicle, repeated-measure ANOVA with Bonferroni correction.

FIG. 12.

12a-b. (a) Immunoprecipitation of the RNA-induced silencing effector complex (RISC) protein, Argonaut 2 (Ago2) in RAA myocytes isolated from patients with AF transiently (48-72 hours) transfected with 20 nM of an locked nucleic acids (LNA)-based miR31 target site blocker (TSB, Exiqon) or with corresponding scrambled control (scr, 20 nM, Exiqon). Further details on the sequence of the TSB and scr control, also see FIG. 13). Molecular weight is shown in kilodalton (kDa); Rb IgG—rabbit immunoglobulin G (as negative control); IP—immunoprecipitation, IB—immunoblotting. (b) miR31 expression is unchanged in Ago2 immunoprecipitates from RAA human myocytes.

12c-f. (c) Sequence of the mature human miR31 (hsa-miR31), 23-nucleotide region of the human DYS-3′UTR, the miR31 hairpin inhibitor (anti-miR31, miRIDIAN, Dharmacon) and the 18-nucleotide-long locked nucleic acids (LNA)-based Target Site Blocker (TSB) covering the miR31-binding site on the DYS-3′UTR (TSB_DYS). (d) Sequences of the mature hsa-miR-31, 23-nucleotide region of the human nNOS-3′UTR, the anti-miR31 (miRIDIAN, Dharmacon) and the 18-nucleotide-long TSB covering the miR31-binding site on the nNOS 3′UTR (TSB_nNOS). The miR-31 seed sequence is in grey. (e, f) Ribonucleoprotein immunoprecipitation with Ago2 in RA myocytes from AF patients. Cells were transfected (48-72 hours) with 20 nM of a LNA-based miR31 TSB for the DYS or nNOS, or corresponding negative controls (NC). Protection of the mir31 binding site with TSB for DYS (n=6 patients) or nNOS (n=7 patients) selectively prevented loading of the respective target mRNA on the RISC complex; #P<0.05 vs NC, 1-way ANOVA with Bonferroni correction. mRNA content of the DYS and nNOS was normalized to the level of miR31 in the RISC complex which was unaltered by the TSB (FIGS. 12a, b). GAPDH mRNA was undetectable in Ago2 precipitates.

12g-m. (g) In the presence of the transcription inhibitor, actinomycin D (5 μg/ml), the presence of miRIDIAN miR31 mimic (10 nM, Dharmacon) accelerated the decay of nNOS mRNA over indicated time course (green filled circles); ###P<0.001 vs scrambled control (empty squares) or untreated cells (in black squares) using2-way ANOVA with Bonferroni correction. By contrast, miR31 mimic has no effect on the decay of the DYS mRNA (h). (I, j) Selective protection of the miR31 binding site on the DYS 3′UTR with LNA-TSB (striped bars, 20 nM, Exiqon) significantly increases both DYS and nNOS protein level (vs NC, n=5 patients/treatment) in human RA myocytes from patients with AF. By contrast, preventing miR31 binding to the nNOS-3′UTR-site 5 with nNOS-TSB (striped bars, 20 nM, Exiqon) recovered nNOS but not DYS (n=5) (i,l); #P<0.05, ###P<0.001 vs NC, 1-way ANOVA with Bonferroni correction. (k, m) By contrast, RA DYS mRNA was unaltered by DYS—TSB (n=8; striped bars), whereas nNOS gene expression was significantly increased in the presence of nNOS-TSB (n=8). ##P<0.01 vs vehicle, 1-way ANOVA with Bonferroni correction. Information on the TSB sequences is summarized in FIG. 13.

FIG. 13.

Sequences of hsa-miR-31-5p mimic and hsa-miR-31-5p hairpin inhibitor (Dharmacon, USA) and locked nucleic acids -based target site blockers (LNA_TSB) (Exiqon).

### EXAMPLES

The following Examples show that an atrial-specific upregulation of miR-31 in AF is responsible for a reduction in dystrophin and syntrophin content in the fibrillating atrial myocardium, leading to nNOS degradation, which is in itself sufficient to recapitulate the hallmark features of AF-induced electrical remodelling. Indeed, nNOS gene deletion or pharmacological inhibition shortens APD90, abolishes the APD rate-dependent adaptation in human and murine atrial myocytes, and significantly increases AF inducibility in response to burst stimulation in mice. miR-31 inhibition fully recovers both dystrophin and nNOS protein content in human atrial myocytes from patients with AF. The predominantly atrial expression of miR-31 and its atrial-selective upregulation in the presence of AF indicate that miR-31 inhibition is a promising new strategy in the treatment of AF.

### Example 1

### Reduction of Nitric Oxide (NO) Bioavailability in AF

In the presence of AF, human (FIG. 1a) and goat (FIG. 1b) atrial samples show a profound reduction in NOS activity (detectable as early as 2 weeks after AF induction in goats, FIG. 1b) that is secondary to a bi-atrial loss of nNOS protein content in atrial myocytes (FIG. 1c-f).

### Example 2

### Effects of Pharmacological Inhibition of nNOS

In mice, nNOS gene deletion has been shown to affect intracellular calcium homeostasis and promote ventricular arrhythmias after myocardial infarction. Here we show that pharmacological inhibition of nNOS with S-methylthiocitrulline (SMTC, 100 nmol/L) shortens action potential duration at 90% repolarization (APD90) and abolishes the APD rate dependent adaptation in right atrial myocytes from patients in sinus rhythm (SR), thereby recapitulating the hallmark features of AF-induced electrical remodelling, but has no effect in atrial myocytes from patients with AF (FIG. 2a, 2b). Similarly, both nNOS gene deletion and pharmacological inhibition shorten atrial APD in mice (FIG. 2c). A shorter APD leads to reduced atrial refractoriness and promote AF sustainability. Accordingly, nNOS−/−mice showed a 2-fold increase in the likelihood of developing AF episodes in response to atrial trans-oesophageal burst pacing (FIG. 2d-f). These findings indicate that loss of myocardial nNOS signalling is sufficient to produce an atrial electrical substrate that increases both the inducibility and maintenance of AF.

### Example 3

### nNOS Gene Expression

Whilst exploring the mechanisms responsible for the loss of atrial nNOS in AF, we found that nNOS gene expression was unaltered (FIG. 3b) but nNOS ubiquitination was significantly increased in the fibrillating atrial myocardium (FIG. 3a). Inhibition of the ubiquitin-proteasome system with MG132 (5 μmol/L) partially restored nNOS content, whereas inhibition of autophagy (with bafilomycin, 25 nmol/L) had no effect (FIG. 3c, 3d). In the skeletal muscle, nNOS protein stability is determined by the enzyme's anchoring to the dystrophin-syntrophin complex. We found a reduction in dystrophin (FIG. 4c, 4d, 4f) and dystrophin-associated proteins (e.g., caveolin-3 and α1-syntrophin, 4c, 4e), and a disruption of the dystrophin-nNOS complex in AF (FIG. 4a, 4b). In humans, atrial nNOS and dystrophin were strongly correlated (FIG. 4g), in keeping with the notion that disruption of the dystrophin-syntrophin complex promotes ubiquitin-dependent nNOS degradation in AF.

### Example 4

### miR-31 Inhibition Restores the Atrial Dystrophin-nNOS Complex in AF

We hypothesised that a microRNA-mediated translational repression of dystrophin in AF may account for the loss of myocardial nNOS and constitute a novel mechanism underlying AF-induced electrical remodelling. Our findings show that miR-31 expression is significantly increased in human right and left atrial homogenates and myocytes in the presence of AF compared to SR (FIG. 5a). Transfection of human atrial myocytes with a miR-31 antagomir (20 nmol/L for 48-72 hours) normalised miR-31 expression to the level observed in SR (FIG. 5b) and restored dystrophin and nNOS content (FIG. 5c, 5d). By contrast, upregulation of miR-31 dramatically reduced both dystrophin and nNOS content in human atrial myocytes from patients in SR (FIG. 5e-g).

These findings indicate that a miR-31 dependent reduction in dystrophin accounts for the loss of nNOS protein in the fibrillating human myocardium.

Importantly, 2 weeks of pacing-induced AF in pigs had no effect on miR-31 expression (FIG. 6a) and nNOS, dystrophin and al-syntrophin protein content in ventricular tissue (FIG. 6f-h), although miR-31 expression was upregulated and the content of all of these proteins was significantly reduced in the atrial myocardium, as observed in goats and human atrial tissue (FIG. 6a-e). In control pigs in SR, ventricular miR-31 expression was 3-fold lower than in the atria (FIG. 6a), in keeping with the results of microRNA sequencing in human ventricular samples, which showed very low levels of miR-31 both in donors hearts and in the presence of hypertrophic or dilated cardiomyopathy. Taken together, these findings demonstrate that inhibition of miR-31 provides a novel atrial-specific strategy in the management of AF and its recurrence.

### Example 5

### Further Effects of miR-31 Inhibition

A significant increase in miR-31 expression was detected in atria homogenates (FIG. 7a) and myocytes from patients with AF (FIG. 7b).

Transfection of atrial myocytes from AF patients with a miR-31 hairpin inhibitor (Dharmacon, 20 nM, 72 hours) decreased miR-31 expression (FIG. 8a) and restored both dystrophin (DYS) and nNOS protein content (FIG. 8b). miR-31 inhibition had no effect on DYS expression but significantly increased nNOS mRNA (FIG. 8b).

By contrast, in atrial myocytes from sinus rhythm (SR) patients, DYS and nNOS protein and mRNA were not significantly altered by miR-31 inhibition (FIG. 9a). In AF myocytes, miR-31 inhibition also restored action potential duration (APD) (reversed by nNOS inhibition, FIG. 9b) and the APD rate-dependent adaptation (FIG. 9c).

Conversely, transfection of SR myocytes with a miRIDIAN miR-31 mimic (Dharmacon, 10 nM, 72 hours,) reduced DYS and nNOS protein content and nNOS mRNA, without affecting DYS expression (FIG. 10a-d). The miR-31 mimic shortened APD and abolished the APD rate-dependent adaptation (FIG. 10e,f).

These findings suggest that, in addition to translational repression of DYS mRNA, miR-31 upregulation in AF may also target nNOS mRNA and hasten its degradation.

Computational analysis predicted five putative miR-31 binding sites in the nNOS 3′UTR (FIG. 11a) only one of which (site 5) was shown to be functional in a reporter assay in HEK 293 cells (FIG. 11b) which also confirmed functionality of the miR-31 binding site in the DYS 3′UTR (FIG. 11c).

Ribonucleoprotein immunoprecipitation with the RNA-induced silencing complex (RISC) protein, Argonaut 2 (Ago2), in AF myocytes showed a reduction in DYS and nNOS mRNA content in the RISC upon blockade of the corresponding miR-31 target site (locked nucleic acids (LNA)-based target site blocker (TSB), 20 nM, Exiqon) (FIG. 12a-f), consistent with a direct interaction of nNOS and DYS mRNA with miR-31. The corresponding mRNA stability was assessed in the presence of a miR-31 mimic (10 nM) in SR myocytes treated with the transcription inhibitor, actinomycin D (5 μg/ml). As shown in FIG. 12g,h, miR-31 accelerated the decay of nNOS mRNA but not of DYS.

Silencing the effect of miR-31 on nNOS in AF myocytes using a TSB (20 nM, Exiqon) increased nNOS mRNA and partially recovered nNOS protein without affecting DYS mRNA and protein content (FIG. 12i-k). By contrast, preventing binding of miR-31 to the DYS 3′UTR restored DYS and partially recovered nNOS protein, in the absence of changes in nNOS or DYS mRNA (FIG. 12i,l,m).

### Example 6

A 65 year old male presents with shortness of breath, reduced exercise capacity, and palpitations. AF is diagnosed. The patient is administered a nucleic acid miR-31 inhibitor (a miR-31 antagomir) by direct injection into the atrial myocardium. Follow-up assessment indicates a significant reduction in AF symptoms and improved quality of life.

1. A nucleic acid that inhibits miR-31 in an atrial myocyte, for use in the prevention or treatment of atrial fibrillation in a subject.

2. The nucleic acid for use according to claim 1, wherein the nucleic acid binds to miR-31.

3. The nucleic acid for use according to claim 2, wherein the nucleic acid comprises a nucleic acid sequence that binds to at least a portion of the miR-31 seed region, wherein said seed region comprises the nucleotides at positions 2-7 of miR-31.

4. The nucleic acid for use according to claim 3, wherein said portion of the miR-31 seed region comprises at least five of the nucleotides at positions 2-7 of miR-31.

5. The nucleic acid for use according to any of claims 2-4, wherein the nucleic acid comprises a nucleic acid sequence that binds to the six nucleotides at positions 2-7 of miR-31.

6. The nucleic acid for use according to any one of claims 2-5, wherein the nucleic acid comprises the nucleic acid sequence CUUGCC.

7. The nucleic acid for use according to any one of claims 2-6, wherein the nucleic acid comprises a nucleic acid sequence having at least 70% sequence identity to SEQ ID NO: 1.

8. The nucleic acid for use according to any of claims 2-7, wherein the nucleic acid is a miR-31 antagomir.

9. The nucleic acid for use according to any of claims 2-7, wherein the nucleic acid is a miR-31 microRNA-sponge.

10. The nucleic acid for use according to claim 1, wherein the nucleic acid competes with miR-31 for binding to a miR-31 mRNA target site in an atrial myocyte.

11. The nucleic acid for use according to claim 10, wherein the nucleic acid comprises the nucleic acid sequence GGCAAG.

12. The nucleic acid for use according to any preceding claim, comprising at least one modified nucleotide; preferably wherein said modified nucleotide is selected from: a locked nucleic acid (LNA) nucleotide, a 2′-O-methyl modified nucleotide, a 2′-O-methoxyethyl modified nucleotide, and a 2′-fluro modified nucleotide.

13. The nucleic acid for use according to claim 12, wherein the nucleic acid consists of modified nucleotides; preferably wherein said modified nucleotides consist of LNA nucleotides, or 2′-O-methyl modified nucleotides, or 2′-O-methoxyethyl modified nucleotides, or 2′-fluro modified nucleotides.

14. The nucleic acid for use according to any one of claims 1-11, wherein the nucleic acid is a nucleic acid analogue selected from: a peptide nucleic acid (PNA), a glycol nucleic acid (GNA), a threose nucleic acid (TNA), and a morpholino.

15. The nucleic acid for use according to any one of claims 1-14, wherein the nucleic acid comprises at least one modified phosphodiester linkage; preferably wherein said modified phosphodiester linkage is a phosphothiorate linkage.

16. A viral vector comprising a nucleic acid sequence encoding a nucleic acid according to any one of claims 1-11, for use in the prevention or treatment of atrial fibrillation in a subject.

17. The viral vector for use according to claim 16, wherein the viral vector is selected from a poxvirus vector, an adenovirus vector, and an adeno-associated virus vector.

18. The nucleic acid for use according to any one of claims 1-15, or the viral vector for use according to claim 16 or 17, wherein the subject experiences a reduction in the symptoms, and/or a reduction in the incidence of complications, of atrial fibrillation.

19. The nucleic acid for use according to claim 18, or the viral vector for use according to 18, wherein the subject experiences one or more of: a reduction in the incidence or recurrence of atrial fibrillation events, a reduction in the severity of an atrial fibrillation event, and a shortening in the duration of an atrial fibrillation event.

20. The nucleic acid for use according to any one of claim 1-15 or 18-19, or the viral vector for use according to claims 16-19, wherein the nucleic acid or viral vector is administered to the subject systemically.

21. The nucleic acid for use according to any one of claim 1-15 or 18-19, or the viral vector for use according to claims 16-19, wherein the nucleic acid or viral vector is administered to the subject by local administration to the heart or a tissue in the vicinity of the heart; preferably wherein the nucleic acid or viral vector is administered to the subject by local administration to the atrial myocardium.

22. The nucleic acid for use according to claim 21, or the viral vector for use according to claim 21, wherein the nucleic acid or viral vector is administered to a coronary artery of the subject.

23. A nucleic acid that inhibits miR-31, for use in a method of increasing nNOS protein levels in the atrial myocardium of a subject.

24. The nucleic acid for use according to claim 23, wherein the nucleic acid is a nucleic acid according to any one of claims 1-15.

25. A pharmaceutical composition for use in the prevention or treatment of atrial fibrillation in a subject, comprising a nucleic acid according to any one of claims 1-15, or a viral vector according to claim 16 or 17, and a pharmaceutically acceptable carrier.

26. A method for diagnosing or predicting the risk of atrial fibrillation in a subject, said method comprising:
(i) determining the amount of miR-31 in a sample from the subject;
(ii) comparing the amount of miR-31 in the sample with a reference standard; and
(iii) identifying a difference in the amount of miR-31 in the sample relative to the reference standard;
wherein an increase in the amount of miR-31 in the sample compared to the reference standard correlates with the presence of, or an increased risk of, atrial fibrillation in the subject; and
wherein a decrease, or no difference in the amount of miR-31 in the sample compared to the reference standard correlates with the absence of an increased risk of atrial fibrillation in the subject.

27. The method of claim 26, wherein the sample is selected from: a blood sample, a plasma sample, a serum sample, and an atrial tissue sample.

28. The method of claim 26 or 27, wherein the amount of miR-31 is determined by measuring the concentration of miR-31 in the sample.


SEQUENCE LISTING (19 sequences)

SEQ ID NO: 1 - RNA, 21 nt, Artificial Sequence
  Nucleic acid sequence complementary to miR-31
agcuaugccagcaucuugccu

SEQ ID NO: 2 - RNA, 21 nt, Artificial Sequence
  Nucleic acid sequence of miR-31
aggcaagaugcuggcauagcu

SEQ ID NO: 3 - RNA, 71 nt, Artificial Sequence
  miR-31 precursor sequence
ggagaggaggcaagaugcuggcauagcuguugaacugggaaccugcuaugccaacauauugccaucuuucc

SEQ ID NO: 4 - RNA, 6 nt, Artificial Sequence
  Target sequence
cuugcc

SEQ ID NO: 5 - RNA, 6 nt, Artificial Sequence
  Target sequence
ggcaag

SEQ ID NO: 6 - DNA, 21 bp, Artificial Sequence
  hsa-miR-31-5p mimic
aggcaagatgctggcatagct

SEQ ID NO: 7 - DNA, 21 bp, Artificial Sequence
  hsa-miR-31-5p hairpin inhibitor
tccgttctacgaccgtatcga

SEQ ID NO: 8 - DNA, 18 bp, Artificial Sequence
  LNA_TSB_nNOS_(site 5)_hsa-miR31-5p
tggcaagaatgaaaaggc

SEQ ID NO: 9 - DNA, 18 bp, Artificial Sequence
  LNA_TSB_dystrophin_hsa-mirR31-5p
ggcaagttatttagctat

SEQ ID NO: 10 - RNA, 24 nt, Artificial Sequence
  NOS1 3′ UTR
nnnnnnnncuggacccucuugccc

SEQ ID NO: 11 - RNA, 21 nt, Artificial Sequence
  hsa-miR-31-5p
ucgauacggucguagaacgga

SEQ ID NO: 12 - RNA, 23 nt, Artificial Sequence
  NOS1 3′ UTR
uguucccacucccucucuugccg

SEQ ID NO: 13 - RNA, 23 nt, Artificial Sequence
  NOS1 3′ UTR
aaauaaaauaaaacaucuugccc

SEQ ID NO: 14 - RNA, 23 nt, Artificial Sequence
  NOS1 3′ UTR
uugaaagaggucaggucuugccc

SEQ ID NO: 15 - RNA, 23 nt, Artificial Sequence
  NOS1 3′ UTR
augcagccuuuucauucuugcca

SEQ ID NO: 16 - DNA, 24 bp, Artificial Sequence
  microRNA mimic negative control
tcacaacctcctagaaagagtaga

SEQ ID NO: 17 - DNA, 24 bp, Artificial Sequence
  microRNA hairpin Inhibitor negative control
tcacaacctcctagaaagagtaga

SEQ ID NO: 18 - DNA, 18 bp, Artificial Sequence
  Negative control for LNA_TSB_nNOS_(site 5)_hsa- miR31-5p
tgacaggaatggaaaagc

SEQ ID NO: 19 - DNA, 19 bp, Artificial Sequence
  Negative control for TSB_Dystrophin_hsa-miR31- 5p
taacacgtctatacgccca